SEQUENCE LISTING (38 sequences)

SEQ ID NO: 1 - protein, 468 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGSAASGSSGGSSSGADIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGSGSGEIVLTQSPGTLSLSPGERATLSCRASQSVSSTYLAWYQQKPGQAPRLLIYGASSRATGIPDRFSGSGSGTDFTLTISRLEPEDFAVYYCQQSGSSPLTFGGGTEVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 454 aa, synthetic construct
QVQLVESGGGVVQPGRSLRLSCAASGFTFSNYGIHWVRQAPGKGLEWVAVIWYDGSIKYYADSVKGRFTISRDNSKNTLYLQMNSLRAEDTAVYYCARDRAAAGLHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 399 aa, synthetic construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGSGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 4 - protein, 447 aa, synthetic construct
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 382 aa, synthetic construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGSGSQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 6 - protein, 477 aa, synthetic construct
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGSAASGSSGGSSSGADIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGSGSGGSGSGGSSGGDIELTQPPSVSVAPGQTARISCSGDNLGKKYVYWYQQKPGQAPVLVIYGDDERPSGIPERFSGSNSGNTATLTISGTQAEDEADYYCASYDSSHILIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 447 aa, synthetic construct
QVQLVESGGGLVQPGGSLRLSCAASGFTFSRYGMHWVRQAPGKGLEWVSGISSIGSNTYYADSVKGRFTISRDNSKNTLYLQMNSLRAEDTAVYYCARWYKTYIDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 379 aa, synthetic construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGSGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVG

SEQ ID NO: 9 - protein, 362 aa, synthetic construct
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGSGSQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVG

SEQ ID NO: 10 - protein, 215 aa, synthetic construct
EIVLTQSPGTLSLSPGERATLSCRASQSVSSTYLAWYQQKPGQAPRLLIYGASSRATGIPDRFSGSGSGTDFTLTISRLEPEDFAVYYCQQSGSSPLTFGGGTEVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 461 aa, synthetic construct
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGSGSGDYKDDDDK

SEQ ID NO: 12 - protein, 453 aa, synthetic construct
QVQLVESGGGVVQPGRSLRLSCAASGFTFSNYGIHWVRQAPGKGLEWVAVIWYDGSIKYYADSVKGRFTISRDNSKNTLYLQMNSLRAEDTAVYYCARDRAAAGLHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 460 aa, synthetic construct
QVQLVESGGGLVQPGGSLRLSCAASGFTFSRYGMHWVRQAPGKGLEWVSGISSIGSNTYYADSVKGRFTISRDNSKNTLYLQMNSLRAEDTAVYYCARWYKTYIDVWGQGTLVTVSSGGGGSGGGGSGGGGSDIELTQPPSVSVAPGQTARISCSGDNLGKKYVYWYQQKPGQAPVLVIYGDDERPSGIPERFSGSNSGNTATLTISGTQAEDEADYYCASYDSSHILIVFGGGTKLTVLGSGSGEIVLTQSPGTLSLSPGERATLSCRASQSVSSTYLAWYQQKPGQAPRLLIYGASSRATGIPDRFSGSGSGTDFTLTISRLEPEDFAVYYCQQSGSSPLTFGGGTEVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 4 aa, synthetic construct
GGGG

SEQ ID NO: 15 - protein, 5 aa, synthetic construct
GGGGG

SEQ ID NO: 16 - protein, 6 aa, synthetic construct
GGGGGG

SEQ ID NO: 17 - protein, 7 aa, synthetic construct
GGGGGGG

SEQ ID NO: 18 - protein, 8 aa, synthetic construct
GGGGGGGG

SEQ ID NO: 19 - protein, 5 aa, synthetic construct
GGGGS

SEQ ID NO: 20 - protein, 4 aa, synthetic construct
GAGA

SEQ ID NO: 21 - protein, 6 aa, synthetic construct
GAGAGA

SEQ ID NO: 22 - protein, 8 aa, synthetic construct
GAGAGAGA

SEQ ID NO: 23 - protein, 4 aa, synthetic construct
AAAA

SEQ ID NO: 24 - protein, 5 aa, synthetic construct
AAAAA

SEQ ID NO: 25 - protein, 6 aa, synthetic construct
AAAAAA

SEQ ID NO: 26 - protein, 7 aa, synthetic construct
AAAAAAA

SEQ ID NO: 27 - protein, 8 aa, synthetic construct
AAAAAAAA

SEQ ID NO: 28 - protein, 6 aa, synthetic construct
GGGGGK

SEQ ID NO: 29 - protein, 7 aa, synthetic construct
GGGGGKR

SEQ ID NO: 30 - protein, 8 aa, synthetic construct
GGGKGGGG

SEQ ID NO: 31 - DNA, 20 bp, synthetic construct
  source 1..20: mol_type = other DNA
ctggaacggtgaaggtgaca

SEQ ID NO: 32 - DNA, 23 bp, synthetic construct
  source 1..23: mol_type = other DNA
aagggacttcctgtaacaatgca

SEQ ID NO: 33 - DNA, 20 bp, synthetic construct
  source 1..20: mol_type = other DNA
atctagtggcttggcatggg

SEQ ID NO: 34 - DNA, 22 bp, synthetic construct
  source 1..22: mol_type = other DNA
ccaaactttcgagtgagccttg

SEQ ID NO: 35 - DNA, 23 bp, synthetic construct
  source 1..23: mol_type = other DNA
cactgaatctgttcttaagcccg

SEQ ID NO: 36 - DNA, 19 bp, synthetic construct
  source 1..19: mol_type = other DNA
ggcgttccacacgtacaga

SEQ ID NO: 37 - DNA, 21 bp, synthetic construct
  source 1..21: mol_type = other DNA
ggaggtgctgaaagcatacct

SEQ ID NO: 38 - DNA, 22 bp, synthetic construct
  source 1..22: mol_type = other DNA
tctcttcagccagtttgaatgc

(19) United States
(12) Patent Application Publication
(10) Pub. No.: US 2026/0365738 A1
(43) Pub. Date: Nov. 19, 2026

(54) CELL TARGETING MULTICOMPONENT POLYPEPTIDE

(71) Applicant: Surrozen Operating, Inc., South San Francisco, CA (US)

(72) Inventor: Yang LI, Mountain View, CA (US)

(21) Appl. No.: 19/103,765

(22) PCT Filed: Aug. 16, 2023
(86) PCT No.: PCT/US2023/072317

§ 371 Date: Feb. 13, 2025

Related U.S. Application Data
(60) Provisional application No. 63/398,502, filed on Aug. 16, 2022.
(60) Provisional application No. 63/472,938, filed on Jun. 14, 2023.

(51) Int. Cl. C07K 16/28

(52) U.S. Cl. CPC C07K 16/2863; C07K 16/28; C07K 2317/31

(57) ABSTRACT

The present invention provides a cell targeting multicomponent polypeptides complexes, to modulate signaling pathways having tissue specificity, by binding to homo- or hetero-oligomeric receptor complexes.

### CROSS REFERENCE TO RELATED APPLICATIONS

This application claims priority to U.S. Provisional Patent Application Ser. No. 63/398,502, filed Aug. 16, 2022, and U.S. Provisional Patent Application Ser. No. 63/472,938, filed Jun. 14, 2023, which are incorporated by reference in their entireties.

### SEQUENCE LISTINGS

The Sequence Listing XML associated with this application is provided in XML file format and is hereby incorporated by reference into the specification. The name of the XML file containing the Sequence Listing XML is SRZN_026_01WO_ST26.xml. The XML file is 39,279 bytes, and created on Aug. 8, 2023, and is being submitted electronically via USPTO Patent Center.

### FIELD OF THE INVENTION

The present disclosure provides cell targeting multicomponent polypeptides that modulate signaling pathways having cell and tissue specificity by binding to hetero-oligomeric receptor complexes.

### BACKGROUND OF THE INVENTION

Receptor-mediated cellular signaling is fundamental to cell-cell communication, cellular growth and differentiation, maintenance of tissue function and homeostasis, and regulation of injury repair. When a cell-specific response is necessary, nature achieves that selectivity through various mechanisms such as cell-specific receptor, ligand gradient, short-range ligand, direct cell-cell contacts. However, most ligand-receptor systems have pleiotropic effects due to broad expressions of receptors on multiple cell types. While this may be important when a coordinated response in numerous cell types or tissues is needed, modulating cell-specific signaling has been a major challenge for research and drug development to avoid systemic toxicity or off-target tissue effects. In addition, it is often difficult to identify differences between normal and diseased cells for a particular signaling pathway of interest, making it challenging, if not impossible, to selectively modulate that signaling pathway either in diseased or normal cells. Therefore, effective cell targeting is needed for both research and therapeutic development.

Cell-targeted antagonists are more accessible to achieve than cell-targeted agonists, as antagonists have less stringent requirements on affinity, epitope, and geometry of the molecules than agonists (Dickopf et al., 2020). Previous efforts to engineer cell selective growth factors and cytokines employed variations of the “chimeric activators” concept (Cironi et al., 2008), which invariably involved attaching a cell targeting arm (“targeting element”) to either the wild-type ligand or a mutated ligand with reduced affinity toward signaling receptor (“activity element”). Such an approach has been applied to several signaling pathways, such as the erythropoietin (Taylor et al., 2010, Burrill et al., 2016), interferon (Cironi et al., 2008, Garcin et al., 2014), and interleukin-2 (Ghasemi et al., 2016, Lazear et al., 2017) pathways.

While little selectivity could be achieved by attaching a targeting arm to a wild-type ligand, selectivity of up to 1000-fold has been reported when a mutated ligand was used (Garcin et al., 2014). The “chimeric activators” approach is based on the cooperativity concept. In this approach, mutations that weaken the affinity of a natural ligand to its receptor are selected as the “activity element”. Due to the weakened affinity, the mutant “activity element” alone should display a significantly right-shifted dose-response curve (or lower potency) in an activity assay on both target and non-target cells that express the signaling receptors.

When a “targeting element” is tethered to the mutant “activity element,” the “targeting element” helps to increase the local concentration of the mutant “activity element” on the desired target cell surface, driving engagement of signaling receptor and subsequent intracellular signaling activation, effectively left shift the activity dose-response curve (increase potency) on the target cell and create the selectivity between target vs. non-target cells.

While conceptually elegant, identifying the appropriate mutations that achieve the just-right reduction of affinity that could be rescued by the “targeting element” is not trivial. In some cases, while the potency could be rescued, the maximal signaling strength remains compromised, resulting in a partial agonist. In addition, the selectivity is not exquisite; a modest 10-fold separation between targeting vs. non-targeting cells is reported in most literature examples.

WNT ligands and their signals play key roles in the control of development, homeostasis and regeneration of many essential organs and tissues, including bone, liver, skin, stomach, intestine, kidney, central nervous system, mammary gland, taste bud, ovary, cochlea and many other tissues (reviewed, e.g., by Clevers, Loh, and Nusse (2014) Science; 346:54). Modulation of WNT signaling pathways has potential for treatment of degenerative diseases and tissue injuries.

The WNT pathway is highly conserved across species and crucial for embryonic development, and adult tissue homeostasis and regeneration (Nusse and Clevers, 2017). WNT-induced signaling through β-catenin stabilization has been widely studied and is achieved by ligand binding to frizzled (FZD) and low-density lipoprotein receptor-related protein (LRP) family of receptors. There are nineteen mammalian WNTs, 10 FZDs (FZD₁₋₁₀), and 2 LRPs (LRP5 and LRP6).

WNTs are highly hydrophobic due to lipidation that is required for function and are promiscuous, capable of binding and activating multiple FZD and LRP pairs (Janda et al., 2012, Kadowaki et al., 1996, Dijksterhuis et al., 2015). Elucidating the functions of individual FZDs in tissues has been hampered by difficulties in producing the ligands and lack of receptor and tissue selectivity. Recent breakthroughs in the development of WNT-mimetic molecules have largely resolved the production and receptor specificity challenges (Janda et al., 2017, Chen et al., 2020, Tao et al., 2019, Miao et al., 2020).

While tissue selectivity could be partly achieved by tissue injury as damaged tissues seem more sensitive to WNTs (Xie et al., 2022), it would still represent a significant technical advancement to be able to target WNTs to specific cells and tissues.

Antibodies are a well-established and rapidly growing drug class with at least 45 antibody-based products currently marketed for imaging or therapy in the United States and/or Europe with ~$100 billion in total worldwide sales. This major clinical and commercial success with antibody therapeutics has fueled much interest in developing the next generation antibody drugs including bispecific antibodies. As their name implies, bispecific antibodies or multispecific antibodies (collectively “MsAbs”) bind to at least two different antigens, or at least two different epitopes on the same antigen, as first demonstrated more than 50 years ago. Engineering monospecific antibodies for multispecificity opens up many new potential therapeutic applications as evidenced by >30 BsAb in clinical development.

Bispecific or multispecific antibodies are a class of engineered antibody and antibody-like proteins that, in contrast to ‘regular’ monospecific antibodies, combine two or more different specific antigen binding elements in a single construct. Since bispecific antibodies do not typically occur in nature, they are constructed either chemically or biologically, using techniques such as cell fusion or recombinant DNA technologies. The ability to bind two or more different epitopes with a single molecule offers a number of potential advantages. One approach is to use the specificity of one arm as a targeting site for individual molecules, cellular markers or organisms, such as bacteria and viruses. While the other arm functions as an effector site for the recruitment of effector cells or delivery of molecular payloads to the target, such as drugs, cytokines or toxins. Alternatively, bispecifics can be used to dual target, allowing detection or binding of a target cell type with much higher specificity than monospecific antibodies.

The modular architecture of immunoglobulins has been exploited to create a growing number (>60) of alternative MsAb formats (see, e.g., Spiess et al (2015) Mol. Immunol. 67:95-106). MsAb are classified here into five distinct structural groups: (i) bispecific IgG (BsIgG) (ii) IgG appended with an additional antigen-binding moiety (iii) MsAb fragments (iv) Multispecific fusion proteins and (v) MsAb conjugates. Each of these different MsAb formats brings different properties in binding valency for each antigen, geometry of antigen-binding sites, pharmacokinetic half-life, and in some cases effector functions.

For antagonistic MsAbs antibodies, which represent the vast majority of the MsAb molecules in development, the geometry of the antigen binding modules is less critical. However, for agonistic MsAbs, these molecules need to faithfully mimic the activity of the natural ligand, the binding geometry could be crucial (see, e.g., Shi, et al. (2018) J. Biol. Chem. 293:5909-5919). Such is true of WNT multicomponent polypeptide molecules, which are required to bind and activate two spatially separated WNT receptors, FZD and LRP.

WNT multicomponent polypeptide molecules which can bind to the hetero-oligomeric WNT/LRP receptor complex have been described previously (see, e.g., WO2019/126398 and WO2020/010308) as have WNT enhancers using RSPO (see, e.g., WO2018/140821, WO2018/132572, and WO2020/014271).

Previous attempts to create multicomponent receptor molecules have been successful for antagonist molecules because lower affinities and tissue selectivity were not as critical. Creating agonist multicomponent receptor molecules has been challenging because of this reduced affinity and lack of selectivity. The present invention addresses this need by the use of a binding composition specific for a bridging molecule, which resides on the cell surface, tethered to binding domains specific for signaling receptor components. This use of a bridging molecule binding domain has provided acceptable affinity for agonist signaling, as well as the ability to confer specificity by target tissue and cell specific cell surface antigens.

### BRIEF DESCRIPTION OF THE DRAWINGS

FIG. 1A shows an optimized design for a WNT mimetic that is a tetravalent bispecific antibody-based molecule.

FIG. 1B shows a cell targeted WNT mimetic based on the “chimeric activator” concept where an “targeting element” is tethered to the WNT mimetic.

FIG. 1C illustrates two separate polypeptides: one FZD binder with additional binding domain that binds “bridging molecule” and one LRP binder that binds “bridging molecule”, where the two molecules bind different epitopes of the “bridging molecule”

FIG. 1D illustrates a WNT mimetic with two FZD binding domains tethered to a bridging molecule by two binding domains specific for two epitopes on the bridging molecule and two LRP binding domains tethered to the bridging molecule by two binding domains specific for two epitopes on the bridging molecule, wherein the two bridging binding epitopes are linked together and each separate molecule could be tethered to “bridging element” by binding to a different epitope on the bridging receptor, creating the final receptor complex.

FIG. 1E shows a WNT mimetic with two FZD binding domain tethered to a bridging molecule by a binding domain specific for a first epitope on the bridging molecule and two LRP binding domains tethered to the bridging molecule by a binding domain specific for a second epitope on the bridging molecule, wherein the two bridging binding epitopes are identical and each separate molecule could be tethered to “bridging element” binding to a epitope on the multimeric bridging receptor, creating the final receptor complex.

FIG. 1F shows a FZD binding domain linked to one LRP binding domain which is tethered to the bridging molecule by a binding domain specific for a first epitope on the bridging molecule and an FZD binding domain linked to one LRP binding domain which is tethered to the bridging molecule by a binding domain specific for a second epitope on the bridging molecule, and each separate molecule could be tethered to a “bridging element” binding to a different epitope on the bridging receptor, creating the final receptor complex.

FIG. 1G shows a FZD binding domain linked to one LRP binding domain which is tethered to the bridging molecule by two binding domains specific for two epitopes on the bridging molecule wherein the two bridging binding epitopes are linked together, and each separate molecule could be tethered to a “bridging element” binding to a different epitope on the bridging receptor, creating the final receptor complex.

FIG. 1H shows FZD binding domain linked to one LRP binding domain which is tethered to the bridging molecule by a binding domain specific for a epitope on the bridging molecule, wherein each separate molecule tethered to a “bridging element” binding to a multimeric bridging receptor, creating the final receptor complex.

FIGS. 2A-2C show selected βKlotho and endocrine fibroblast growth factor 21 (FGF21) ligand system as the bridging receptor system, with the FZD and LRP binding domains. The graphic representations of the binders and the various combinations F12578 (binds FZD1,2,5,7,8) and L (binds LRP5/6). FIG. 2A shows elements that are used in the split molecules. LRP6 binder element is in scFv format; FZD binder is in the IgG1 format; both αGFP IgG1 and αGFP scFv are used for assembly of the negative control molecules. Two types of βKlotho binders are used: Binder No. 1, 39F7 IgG1, a βKlotho monoclonal antibody; Binder No. 2, FGF21FL, and different deletion variants. FIG. 2B illustrates the diagram of the assembled molecules. FIG. 2C shows the diagram of the assembled negative control molecules.

FIGS. 2D-2G show binding of various F-FGF21 fusion proteins to FZD7 and βKlotho. Bindings of FZD7 and β-Klotho to F-FGF21FL (FIG. 2D), F-FGF21ΔN (FIG. 2E), F-FGF21ΔC (FIG. 2F), or F-FGF21ΔNΔC (FIG. 2G) were determined by Octet.

FIGS. 2H-2J illustrate binding of LRP6 and βKlotho to L-39F7 (FIG. 2H), αGFP-39F7 (FIG. 2I), or L-αGFP (FIG. 2J) were determined by Octet. Mean KD values were calculated from all 7 binding curves with global fits (red dotted lines) using 1:1 Langmuir binding model.

FIGS. 2K-2L show step bindings, FIG. 2K is step binding of FZD7 and βKlotho to various F-FGF21 fusion proteins. Sequential binding of F-FGF21FL (blue sensorgram), F-FGF21ΔN (red sensorgram), F-FGF21ΔC (light green sensorgram), or F-FGF21ΔNΔC (green sensorgram), followed by FZD7 CRD, then followed by addition of Klotho on Octet shows simultaneous engagement of both FZD7 and Klotho to the indicated F-FGF21 proteins. Sensorgrams for FZD7 and βKlotho area (dashed box on left) are enlarged at the right. FIG. 2L shows step binding of LRP6 and βKlotho to L-39F7 and its control proteins. Sequential binding L-39F7 (blue sensorgram), L-αGFP (light blue sensorgram), αGFP-39F7 (red sensorgram), or 39F7 IgG (green sensorgram), followed by LRP6E3E4, then followed by addition of βKlotho on Octet shows simultaneous engagement of both LRP6 and βKlotho to the indicated L-39F7 and its control proteins. Sensorgrams for LRP6 and βKlotho area (dashed box on left) are enlarged on the right.

FIGS. 3A-3F illustrate dose dependent STF assay of SWIFT molecules in HEK293 and Huh7 cells the combination of F12578-FGF21FL or FGF12578-FGF21ΔN with L-39F7 resulted in WNT/β-catenin signaling in a liver cell line, Huh7 cells, which express the bridging receptor βKlotho, but not in 293 cells where βKlotho is not expressed. This signaling depends on the presence of both FZD and LRP binding arms and the ability to bind the bridging receptor, as the removal of LRP binding arm L from L-39F7 or inactivation of Klotho binding (use of FGF21ΔNΔC) resulted in no activity in either cell.

FIG. 3G illustrates expression of bridging receptor βKlotho (KLB) in HEK293 and Huh7 cells.

FIGS. 4A and 4B show the FZD binder (F12578), LRP binder (L), and the two non-competing bridging receptor (βKlotho) binders (FGF21FL and 39F7) were combined in the molecule, F12578-FGF21FL-39F7-L (FIG. 4A), and their activity WNT/β-catenin signaling in Huh7 cells that expresses the bridging receptor βKlotho but not in 293 cells where βKlotho is not expressed (FIG. 4B).

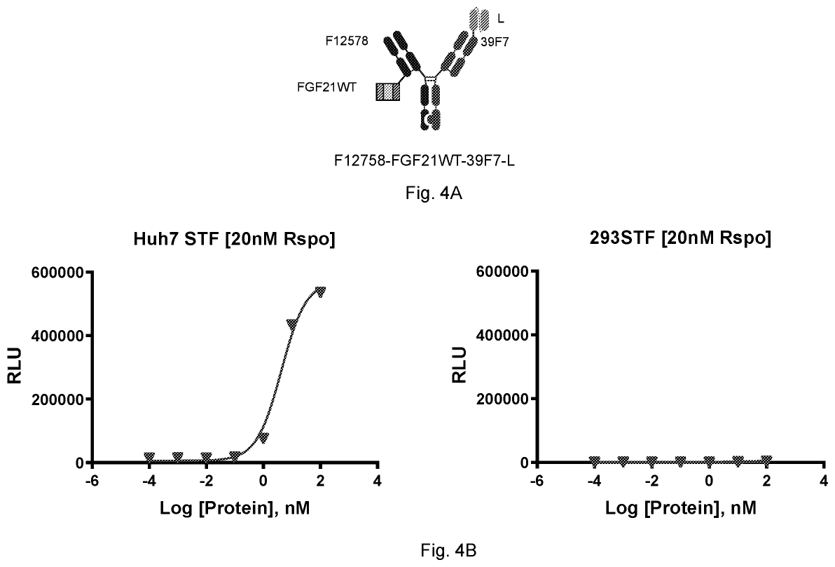

FIGS. 5A-5D illustrate activity of targeted molecules in primary human cells. FIG. 5A is presentative images of primary human hepatocytes cultures in 2D or human small intestinal organoids. Scale bars 200 μm. FIG. 5B shows expression of bridging receptor βKlotho (KLB) in hepatocytes or small intestinal cells. FIG. 5C illustrates WNT target gene AXIN2 expression normalized to control treatment after 24-hour treatment with 10 nM of indicated molecules in human hepatocytes. FIG. 5D shows WNT target gene AXIN2 expression normalized to control treatment after 24-hour treatment with 10 nM of indicated molecules in human small intestinal organoids.

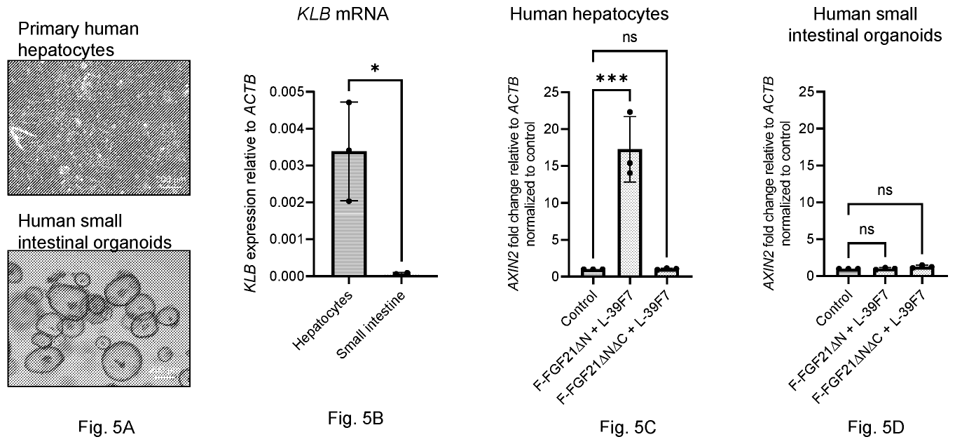

### SUMMARY OF THE INVENTION

In various embodiments, the present invention provides a cell targeting multicomponent polypeptide and related uses thereof.

The present invention provides a cell targeting multicomponent polypeptide molecule comprising at least one first antigen binding domain that binds to a first signaling receptor component, at least one-second antigen binding domain that binds to a second signaling receptor component receptor, and at least one different antigen binding domain that binds to a bridging molecule, and the first receptor and second receptor can be different or identical.

In some embodiments, the binding domain that binds to the bridging molecule comprises at least two binding domains that bind to different epitopes on the bridging molecule or bind to the same epitope on the bridging molecule.

In another embodiment, the first antigen binding domain that binds to a first signaling receptor component is tethered to at least one binding domain that binds to the bridging receptor, and the second antigen binding domain that binds to a second signaling receptor component is tethered to at least one binding domain that binds to the bridging molecule.

In a further embodiment, the antigen binding domain is joined directly or by a linker to the binding domain that binds to the bridging molecule.

In another embodiment, the bridging molecule is monomeric or multimeric.

In a further embodiment, the binding domains are each independently selected from the group consisting of: an scFv, a VHH/sdAb, a Fab, and a Fab′2.

In some embodiment, the binding domains are joined through a linker by a peptide linker comprising from 1-100 amino acids.

In yet another embodiment, the binding domains are attached to the N-terminus of an antibody Fc domain. In another embodiment, a nucleic acid encodes the multicomponent polypeptide.

In a further embodiment, the nucleic acid is an expression vector comprises a promoter sequence operatively linked to the nucleic acid sequence; optionally wherein the vector is a virus comprising a promoter operatively linked to the nucleic acid.

In another embodiment, a host cell comprises the vector.

In related embodiment provides a process for producing the multicomponent polypeptide comprising culturing the host cell under conditions wherein the multicomponent polypeptide is expressed by the expression vector.

The present invention further provides a multicomponent WNT molecule comprises at least one FZD binding domain, at least one LRP binding domain, and at least one bridging molecule; the multicomponent WNT multicomponent polypeptide molecule modulates WNT signaling.

In yet another embodiment, the multicomponent WNT molecule comprises: one or two FZD binding domains tethered to the bridging molecule by a binding domain specific for a first epitope on the bridging molecule; one or two LRP binding domains tethered to the bridging molecule by a binding domain specific for a second epitope on the bridging molecule, and the molecule can activate WNT signaling.

In some embodiments, the multicomponent WNT molecule comprises the binding domain specific for the first epitope of the bridging molecule and the FZD binding domain(s) are joined directly or joined by a linker; and the binding domain specific for the second epitope of the bridging molecule and the LRP binding domain(s) are joined directly or joined by a linker; the binding domain specific for the first epitope and the binding domain specific for the second epitope of the bridging molecule are identical or different.

In another embodiment, the linker is a peptide or non-peptide linker.

In yet another embodiment, the FZD binding domain(s) and the LRP binding domain(s) are each independently selected from the group consisting of: a scFv, a VHH/sdAb, a Fab, and a Fab′2.

In a further embodiment, the FZD binding domain(s) and the LRP binding domains(s) are joined by a peptide linker comprising from 1-100 amino acids.

In yet another embodiment, the FZD binding domain(s) and the LRP binding domain(s) are attached to the N-terminus of an antibody Fc domain.

In some embodiment, a nucleic acid encoding the multicomponent WNT molecule.

In another embodiment, the vector is an expression vector comprises a promoter sequence operatively linked to the nucleic acid sequence; optionally wherein the vector is a virus comprising a promoter operatively linked to the nucleic acid.

In a further embodiment, a host cell comprises the vector.

In some embodiments, a process for producing the multicomponent WNT molecule culturing the host cell under conditions wherein the multicomponent polypeptide is expressed by the expression vector.

In some embodiment, a pharmaceutical composition to modulate the WNT/β-catenin signaling pathway comprises an effective amount of the multicomponent WNT molecule, and a pharmaceutically acceptable diluent, adjuvant, or carrier.

In yet another embodiment, provides a method of treating or preventing a disease or disorder in a subject in need thereof, the method comprises by providing to the subject an effective amount of the multicomponent WNT molecule.

The present invention also provides a multicomponent WNT molecule comprises at least one FZD binding domain linked to one LRP binding domain which is tethered to the bridging molecule by a binding domain specific for a first epitope on the bridging molecule; at least one FZD binding domain linked to one LRP binding domain which is tethered to the bridging molecule by a binding domain specific for a second epitope on the bridging molecule; and the tethering of the FZD and LRP binding domains to the bridging receptor forms a WNT mimetic capable of activating WNT signaling.

In a further embodiment, the binding domain specific for a first epitope of the bridging molecule and the FZD/LRP binding domains are joined directly or by a linker; and the binding domain specific for a second epitope of the bridging molecule and the FZD/LRP binding domains are joined directly, or by a linker the binding domain of the first epitope and second epitope of the bridging molecule are identical or different; and the binding domain of the first epitope and the second epitope of the bridging molecule could be linked together to FZD/LRP.

In some embodiments, the bridging molecule is monomeric or multimeric.

In another embodiment, at least one of the FZD or LRP binding domains is selected from the group consisting of: a scFv, a VHH/sdAb, a Fab, and a Fab′2.

In yet another embodiment, the binding domains are joined through a linker by a peptide link comprising from 1-100 amino acids.

In related embodiment, the binding domains are attached to the N-terminus of an antibody Fc domain.

In some embodiment, a nucleic acid encoding the multicomponent WNT molecule.

In another embodiment, the vector is an expression vector comprises a promoter sequence operatively linked to the nucleic acid sequence; optionally wherein the vector is a virus comprising a promoter operatively linked to the nucleic acid.

In a further embodiment, a host cell comprises the vector.

In some embodiments, a process for producing the multicomponent WNT molecule culturing the host cell under conditions wherein the multicomponent polypeptide is expressed by the expression vector.

In some embodiment, a pharmaceutical composition to modulate the WNT/β-catenin signaling pathway comprises an effective amount of the multicomponent WNT molecule, and a pharmaceutically acceptable diluent, adjuvant, or carrier.

In yet another embodiment, provides a method of treating or preventing a disease or disorder in a subject in need thereof, the method comprises by providing to the subject an effective amount of the multicomponent WNT molecule.

### DETAILED DESCRIPTION

As used herein, including the appended claims, the singular forms of words such as “a,” “an,” and “the,” include their corresponding plural references unless the context clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

All references cited herein are incorporated by reference to the same extent as if each individual publication, patent application, or patent, was specifically and individually indicated to be incorporated by reference.

### I. DEFINITIONS

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “tethered” or “bound” or “joined” or “linked”, as used herein, refer to different binding domains of the multicomponent molecule being fastened together. It is understood that different binding domains of the multicomponent molecule are fastened together and could be used interchangeably.

The terms “targeting element”, “bridging element”, “bridging molecule”, or “bridging receptor” as used herein, refer to cell surface receptors also known as transmembrane receptors, such as, e.g., ion channel-linked receptors, G-protein coupled receptors, and enzyme-linked receptors. A bridging receptor can be a cell surface glycan.

The terms “chimeric activator”, as used herein, refers to the cooperativity concept of different ligands by enhancing agonist activity to the targeted cell.

The terms “multicomponent polypeptide”, “multicomponent polypeptide molecule”, and “multicomponent molecule”, as used herein, are interchangeable.

The terms “multicomponent WNT molecule”, “WNT multicomponent polypeptide molecule”, and “WNT mimetic” as used herein, are interchangeable.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As is well known in the art, an antibody is an immunoglobulin molecule capable of specific binding to a target, such as a carbohydrate, polynucleotide, lipid, polypeptide, etc., through at least one epitope recognition site, located in the variable region of the immunoglobulin molecule. As used herein, the term encompasses not only intact polyclonal or monoclonal antibodies, but also fragments thereof (such as dAb, Fab, Fab′, F(ab′)2, Fv), single chain (scFv), VHH, synthetic variants thereof, naturally occurring variants, fusion proteins comprising an antibody or an antigen-binding fragment thereof, humanized antibodies, chimeric antibodies, and any other modified configuration of the immunoglobulin molecule that comprises an antigen-binding site or fragment (epitope recognition site) of the required specificity. “Diabodies”, multivalent or multispecific fragments constructed by gene fusion (WO94/13804; P. Holliger et al (1993), Proc. Natl. Acad. Sci. USA 90 6444-6448) are also a particular form of antibody contemplated herein. Minibodies comprising a scFv joined to a CH3 domain are also included herein (See e.g., S. Hu et al. (1996), Cancer Res., 56:3055-3061; Ward, E. S. et al. (1989) Nature 341:544-546; Bird et al. (1988) Science 242:423-426; Huston et al. (1988) Proc. Natl. Acad. Sci. USA 85:5879-5883; PCT/US92/09965; WO94/13804; P. Holliger et al. (1993) Proc. Natl. Acad. Sci. USA 90:6444-6448; and Y. Reiter et al. (1996) Nature Biotech. 14:1239-1245).

The term “antigen-binding fragment” as used herein refers to a polypeptide fragment that contains at least one CDR of an immunoglobulin heavy and/or light chain, or of a VHH, that binds to the antigen of interest, in particular to one or more FZD receptor or LRP5 or LRP6 receptor. In this regard, an antigen-binding fragment of the herein described antibodies may comprise 1, 2, 3, 4, 5, or all 6 CDRs of a VH and VL sequence set forth herein from antibodies that bind one or more FZD receptor or LRP5 and/or LRP6. In particular embodiments, an antigen-binding fragment may comprise all three VH CDRs or all three VL CDRs. Similarly, an antigen binding fragment thereof may comprise all three CDRs of a VHH binding fragment. An antigen-binding fragment of a FZD-specific antibody is capable of binding to a FZD receptor. An antigen-binding fragment of a LRP5/6-specific antibody is capable of binding to a LRP5 and/or LRP6 receptor. As used herein, the term encompasses not only isolated fragments but also polypeptides comprising an antigen-binding fragment of an antibody disclosed herein, such as, for example, fusion proteins comprising an antigen-binding fragment of an antibody disclosed herein, such as, e.g., a fusion protein comprising a VHH that binds one or more FZD receptors and a VHH that binds LRP5 and/or LRP6.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a multicomponent WNT molecule or binding region thereof) is said to specifically bind an antigen when it preferentially recognizes its target antigen in a complex mixture of proteins and/or macromolecules. In certain embodiments, a multicomponent WNT molecule or binding region thereof (e.g., an antibody or antigen-binding fragment thereof) is said to specifically bind an antigen when the equilibrium dissociation constant is ≤10⁻⁷ or ≤10⁻⁸ M. In some embodiments, the equilibrium dissociation constant may be ≤10⁻⁹ M or ≤10⁻¹⁰ M.

As used herein, the term “CDR” refers to at least one of the three hypervariable regions of a heavy or light chain variable (V) region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site. In certain embodiments, antibodies and antigen-binding fragments thereof as described herein include a heavy chain and a light chain CDRs, respectively interposed between a heavy chain and a light chain framework regions (FRs) which provide support to the CDRs and define the spatial relationship of the CDRs relative to each other.

As used herein, the term “FRs” refer to the four flanking amino acid sequences which frame the CDRs of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains. The structures and locations of immunoglobulin CDRs and variable domains may be determined by reference to Kabat, E. A. et al., Sequences of Proteins of Immunological Interest. 4th Edition. US Department of Health and Human Services. 1987, and updates thereof, now available on the Internet (immuno.bme.nwu.edu).

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), V_{HH}, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including multicomponent WNT molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”

The term “co-receptor” refers to a first cell surface receptor that binds signaling molecule or ligand in conjunction with another receptor to facilitate ligand recognition and initiate a biological process, such as WNT pathway signaling.

The term “agonist activity” refers to the ability of an agonist to mimic the effect or activity of a naturally occurring protein.

As used herein “peptide linker” or “linker moiety” refers to a sequence of sometimes repeating amino acid residues, usually glycine and serine, that are used to join the various antigen binding domains described below. The length of the linker sequence determines the flexibility of the antigen binding domains in MsAbs, in particular, in the binding of epitopes on co-receptors such as FZD receptors, LRP5 and/or LRP6, and/or ZNRF3/RNF43.

As used herein, the term “enhances” refers to a measurable increase in the level of receptor signaling modulated by a ligand or ligand agonist compared with the level in the absence of the agonist, e.g., a multicomponent WNT molecule. In particular embodiments, the increase in the level of receptor signaling is at least 10%, at least 20%, at least 50%, at least two-fold, at least five-fold, at least 10-fold, at least 20-fold, at least 50-fold, or at least 100-fold as compared to the level of receptor signaling in the absence of the agonist, e.g., in the same cell type.

An antigen or epitope that “specifically binds” or “preferentially binds” (used interchangeably herein) to an antibody or antigen-binding fragment thereof is a term well understood in the art, and methods to determine such specific or preferential binding are also well known in the art. A molecule, e.g., a multicomponent WNT molecule, is said to exhibit “specific binding” or “preferential binding” if it reacts or associates more frequently, more rapidly, with greater duration and/or with greater affinity with a particular cell or substance than it does with alternative cells or substances. A molecule or binding region thereof, e.g., a multicomponent WNT molecule or binding region thereof, “specifically binds” or “preferentially binds” to a target antigen, e.g., a FZD receptor, if it binds with greater affinity, avidity, more readily, and/or with greater duration than it binds to other substances. It is also understood by reading this definition that, for example, a multicomponent WNT molecule or binding region thereof that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al. (1986) Nucl. Acids Res. 14:9081; Stec et al. (1984) J. Am. Chem. Soc. 106:6077; Stein et al. (1988) Nucl. Acids Res. 16:3209; Zon et al. (1991) Anti-Cancer Drug Design, 6:539; Zon et al. (1991) Oligonucleotides and Analogues: A Practical Approach, pp. 87-108 (F. Eckstein, Ed.), Oxford University Press, Oxford England; Stec et al., U.S. Pat. No. 5,151,510; Uhlmann and Peyman (1990) Chem. Rev. 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

The term “host cell” is used to refer to a cell into which has been introduced, or which is capable of having introduced into it, a nucleic acid sequence encoding one or more of the herein described polypeptides, and which further expresses or is capable of expressing a selected gene of interest, such as a gene encoding any herein described polypeptide. The term includes the progeny of the parent cell, whether or not the progeny are identical in morphology or in genetic make-up to the original parent, so long as the selected gene is present. Accordingly, there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g., chromosome) of the host cell. Integration may be promoted by inclusion of sequences which promote recombination with the genome, in accordance-with standard techniques.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, MOLECULAR CLONING, A LABORATORY MANUAL, Cold Spring Harbor Laboratories; Davis et al., 1986, BASIC METHODS IN MOLECULAR BIOLOGY, Elsevier; and Chu et al., 1981, Gene 13:197. Such techniques can be used to introduce one or more exogenous DNA moieties into suitable host cells.

“Transduction” refers to the acquisition and transfer of eukaryotic cellular sequences by viruses, e.g., retroviruses.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell.

The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

The terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass multicomponent WNT molecules, FZD binding regions thereof, LRP5/6 binding regions thereof, antibodies and antigen-binding fragments thereof that bind to a FZD receptor or a LRP5 or LRP6 receptor disclosed herein, or sequences that have deletions from, additions to, and/or substitutions of one or more amino acid of any of these polypeptides. Thus, a “polypeptide” or a “protein” can comprise one (termed “a monomer”) or a plurality (termed “a multimer”) of amino acid chains. Polypeptides and proteins include glycoproteins.

The term “isolated protein,” “or “isolated antibody” referred to herein means that a subject protein, multicomponent molecule, or antibody: (1) is free of at least some other proteins with which it would typically be found in nature; (2) is essentially free of other proteins from the same source, e.g., from the same species, (3) is expressed by a cell from a different species; (4) has been separated from at least about 50 percent of polynucleotides, lipids, carbohydrates, or other materials with which it is associated in nature; (5) is not associated (by covalent or noncovalent interaction) with portions of a protein with which the “isolated protein” is associated in nature; (6) is operably associated (by covalent or noncovalent interaction) with a polypeptide with which it is not associated in nature; or (7) does not occur in nature. Such an isolated protein can be encoded by genomic DNA, cDNA, RNA or other RNA, or may be of synthetic origin, or any combination thereof. In certain embodiments, an isolated protein may comprise naturally-occurring and/or artificial polypeptide sequences. In certain embodiments, the isolated protein is substantially free from proteins or polypeptides or other contaminants that are found in its natural environment that would interfere with its use (therapeutic, diagnostic, prophylactic, research or otherwise).

A “WNT super agonist” is a molecule having enhanced WNT agonist activity. As used herein, the WNT super agonists have both WNT signaling and WNT signal enhancing activity. In some embodiments, the WNT super agonist molecule will bind both at least one FZD receptor and at least one LRP receptor, as well as binding and activating at least one E3 ubiquitin ligase receptor, thereby stabilizing the FZD and/or LRP receptors.

### II. GENERAL

The present invention provides combinations of antigen binding molecules that act as multicomponent polypeptide and enhancing molecules by binding to and modulating co-receptor signaling, for example, antigen binding molecules that bind to one or more FZD receptor and one or more LRP5 or LRP6 receptor, and a bridging receptor, which in turn modulates a downstream WNT signaling pathway. In particular embodiments, the multicomponent complex activates or increases a signaling pathway associated with one or both co-receptors by homo- or hetero-dimerization after interaction of a bridging element with the bridging receptor. In particular embodiments, the multicomponent polypeptide molecules disclosed herein comprise: (i) one or more antibodies or antigen-binding fragments thereof that specifically bind to one or more first co-receptor, including antibodies or antigen-binding fragments thereof having particular co-receptor specificity and/or functional properties; and (ii) one or more antibodies or antigen-binding fragments thereof that specifically bind to one or more second co-receptors, and one or more bridging molecule that bind to one or more bridging receptor. Certain embodiments encompass specific structural formats or arrangements of the first and second co-receptor binding region(s) of the multicomponent molecules advantageous in increasing downstream signaling and related biological effects.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Sequences of illustrative antibodies, or antigen-binding fragments, or complementarity determining regions (CDRs) thereof, that bind to one or more FZD receptors, are set forth in WO2019126399. Sequences of illustrative LRP5 and/or LRP6 antibodies, or antigen-binding fragments, or complementarity determining regions (CDRs) thereof, are set forth in WO2019126401. Sequences of antigen binding molecules that bind one or more FZD receptor and LRP5 and/or LRP6 are set forth in U.S. Provisional application Nos. 62/607,875, 62/641,217, and 62/680,522, titled WNT Signaling Pathway Agonists, filed Dec. 19, 2017, Mar. 9, 2018, and Jun. 4, 2018, respectively.

Antibodies and antibody fragments thereof may be prepared by methods well known in the art. For example, the proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH: VL heterodimer including an antigen-binding site which retains much of the antigen recognition and binding capabilities of the native antibody molecule. (See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096).

In certain embodiments, single chain Fv or scFV antibodies are contemplated. For example, Kappa bodies (Ill et al. (1997), Prot. Eng. 10:949-57; minibodies (Martin et al. (1994) EMBO J 13:5305-9; diabodies (Holliger et al. (1993) PNAS 90:6444-8; or janusins (Traunecker et al. (1991) EMBO J 10:3655-59; and Traunecker et al. (1992) Int. J. Cancer Suppl. 7:51-52), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more FZD receptor through one binding domain and to a second molecule through a second binding domain and bridging molecule with identical or different bridging epitopes for the first or second binding domains. These antibodies may be produced through recombinant molecular biological techniques or may be physically conjugated together.

A single chain Fv (scFv) polypeptide is a covalently linked VH::VL heterodimer which is expressed from a gene fusion including VH- and VL-encoding genes linked by an encoded peptide linker. Huston et al. (1988) Proc. Nat. Acad. Sci. USA 85 (16): 5879-5883. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). A dAb fragment of an antibody consists of a VH domain (Ward, E. S. et al. (1989) Nature 341:544-546).

Where bispecific antibodies are to be used, these may be conventional bispecific antibodies, which can be manufactured in a variety of ways (Holliger, P. and Winter G. (1993) Curr. Op. Biotechnol. 4:446-449), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al. (1996) Protein Eng., 9:616-621).

In certain embodiments, the antibodies described herein may be provided in the form of a UniBody®. A UniBody® is an IgG4 antibody with the hinge region removed (see GenMab Utrecht, The Netherlands; see also, e.g., US20090226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, the antibodies of the present disclosure may take the form of a single variable domain fragment known as a V_{HH}. The V_{HH} technology was originally developed following the discovery and identification that camelidae (e.g., camels and llamas) possess fully functional antibodies that consist of heavy chains only and therefore lack light chains. These heavy-chain only antibodies contain a single V_{HH} domain and two constant domains (CH2, CH3). The cloned and isolated V_{HH} domains have full antigen binding capacity and are very stable. These V_{HH} domains are encoded by single genes and are efficiently produced in almost all prokaryotic and eukaryotic hosts e.g., E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). The production process is scalable and multi-kilogram quantities of VHHs have been produced. VHHs may be formulated as a ready-to-use solution having a long shelf life. The Nanoclone® method (see, e.g., WO 06/079372) is a proprietary method for generating VHHs against a desired target, based on automated high-throughput selection of B-cells. VHH antibodies typically have a small size of around 15 kDa.

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al. (1988) Proc Natl Acad Sci USA 86:10029-10033; and Riechmann et al. (1988) Nature 332:323-327). Illustrative methods for humanization of the anti-FZD antibodies disclosed herein include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856. Riechmann, L., et al., (1988) supra; Verhoeyen, M., et al., (1988) Science 239:1534-1536; Kettleborough, C. A., et al., (1991) Protein Engg 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio/Technol. 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

### III. STRUCTURES OF MULTICOMPONENT RECEPTOR COMPLEXES

The disclosure provides, in certain aspects, a cell targeting multicomponent polypeptide having a one or more first antigen binding domain that binds to a first receptor, a one or more second antigen binding domain that binds to a second receptor, and one or more different antigen binding domain that binds to one or more bridging molecule that activates the signaling pathways.

In certain illustrative embodiments of the tissue-specific bridging molecules disclosed herein: the tissue is bone tissue, and the cell surface receptor is parathyroid hormone receptor 1 (PTH1R); or the tissue is liver tissue, and the cell surface receptor is asialoglycoprotein receptor 1 (ASGR1), asialoglycoprotein receptor 2 (ASGR2), transferrin receptor 2 (TFR2) or solute carrier family 10 member 1 (SLC10A1), or the tissue is oral mucous tissue, and the cell surface receptor is LY6/PLAUR Domain Containing 3 (LYPD3) or Desmoglein 3 (DSG3).

In certain illustrative embodiments of the tissue-specific bridging molecules disclosed herein: the cell surface molecule is a PTH1, and at least one different antigen binding domain specifically binds PTH1R; the cell surface molecule is ASGR1, and at least one different antigen binding domain specifically binds ASGR1; the cell surface molecule is ASGR2, and third binding at least one different antigen binding domain binds ASGR2; the cell surface molecule is SLC10A1, and at least one different antigen binding domain specifically binds SLC10A1; or the cell surface molecule is TFR2, and at least one different antigen binding domain specifically binds TFR2, the cell surface molecule is LYPD3, and at least one different antigen binding domain specifically binds LYPD3; or the cell surface molecule is DSG3, at least one different antigen binding domain sequence specifically binds DSG3, at least one different antigen binding domain is an antibody or fragment thereof, a small molecule, or a ligand, or fragment or variant thereof, of the cell surface molecule.

In certain embodiment, the tethering of the targeting element or the bridging molecule to the activity element or the ensemble of the first antigen binding domain that binds to a first receptor, a one or more second antigen binding domain that binds to a second receptor, increase the local concentration of the activity element on the desired target cell surface, by engagement of signaling receptor and subsequent intracellular signaling activation. Thus, the tethering of the first and second binding domains to the bridging receptor forms a chimeric activator capable of activating the targeted cell signaling.

In certain embodiments, a multicomponent molecule is capable of modulating signaling events associated with at least one of the co-receptors that it binds, in a cell contacted with the multicomponent polypeptide molecule. In certain embodiments, the multicomponent polypeptide molecule increases receptor signaling. As an example, a WNT multicomponent polypeptide molecule specifically modulates the biological activity of a human WNT/β-catenin signaling pathway.

Multicomponent polypeptide molecules of the present invention are biologically active in binding to one or more of a first receptor and to one or more of a second receptor, and as an example, in the activation of WNT signaling, the WNT multicomponent polypeptide molecule is a WNT agonist. The term “agonist activity” refers to the ability of an agonist to mimic the effect or activity of a naturally occurring protein binding to a first and second receptor. The ability of the multicomponent polypeptide molecules and other receptor agonists disclosed herein to mimic the activity of the natural ligand can be confirmed by a number of assays. As an example, WNT multicomponent polypeptide molecules, some of which are disclosed herein activate, enhance or increase the canonical WNT/β-catenin signaling pathway.

In particular embodiments, the structures of the multicomponent polypeptide molecules disclosed herein are bispecific, i.e., they specifically bind to two or more different epitopes, e.g., one or more epitopes of a first receptor, and one or more epitopes of a second receptor.

In particular embodiments, multicomponent molecules disclosed herein are multivalent, e.g., they comprise two or more regions that each specifically bind to the same epitope, e.g., two or more regions that bind to an epitope within one or more first co-receptor and/or two or more regions that bind to an epitope within a second co-receptor. In particular embodiments, they comprise two or more regions that bind to an epitope within a first co-receptor and two or more regions that bind to an epitope within a second co-receptor. In certain embodiments, multicomponent polypeptide molecules comprise a ratio of the number of regions that bind one or more first co-receptor to the number of regions that a second co-receptor of or about: 1:1, 2:1, 3:1, 4:1, 5:1, 6:1, 2:3, 2:5, 2:7, 7:2, 5:2, 3:2, 3:4, 3:5, 3:7, 3:8, 8:3, 7:3, 5:3, 4:3, 4:5, 4:7, 4:9, 9:4, 7:4, 5:4, 6:7, 7:6, 1:2, 1:3, 1:4, 1:5, or 1:6. In certain embodiments, the multicomponent polypeptide molecules are bispecific and multivalent.

In particular embodiment, the multicomponent WNT molecule described herein, are bispecific and can simultaneously bind to FZDs and LRPs binding domains. The optimal stoichiometry (at least for the antibody based molecules) are tetravalent bispecific (2:2 format), requiring two FZD binders and two LRP binders in the same molecule to achieve efficient signaling (Tao et al., 2019, Chen et al., 2020). Taking advantage of the fact that two FZD binders alone, two LRP binders alone, or a molecule with one FZD binder and one LRP binder (1:1 format) do not signal. Cell specificity is achieved by attaching an “targeting element” (capable of binding to another cell surface receptor, called a bridging receptor here) to these three inactive molecules, and signaling competent receptor complexes consisting of two FZDs and two LRPs could then be assemble via the bridging receptor on the target cell surface. This approach reduces or eliminates the need to mutate and reduce the affinity of the “active elements” toward signaling receptors, and creates a highly cell specific activation of the signaling pathway as the individual components are inactive. In particular embodiments, the antigen binding domain(s) binds to a signaling receptor or a signaling receptor component.

The structures of the multicomponent polypeptide molecules disclosed herein may have any of a variety of different structural formats or configurations. The multicomponent polypeptide molecules may comprise polypeptides and/or non-polypeptide binding moieties, e.g., small molecules. In particular embodiments, the multicomponent polypeptide molecules comprise both a polypeptide region and a non-polypeptide binding moiety. In certain embodiments, the multicomponent polypeptide molecules may comprise a single polypeptide, or they may comprise two or more, three or more, or four or more polypeptides.

In certain embodiments, one or more polypeptides of a multicomponent polypeptide molecule are antibodies or antigen-binding fragments thereof. In certain embodiments, surrogates comprise two antibodies or antigen binding fragments thereof, one that binds one or more first co-receptor and one that binds on or more second co-receptor. In certain embodiments, the molecules, e.g., surrogates, comprise one, two, three, or four polypeptides, e.g., linked or bound to each other or fused to each other.

When the multicomponent polypeptide molecules comprise a single polypeptide, they may be a fusion protein comprising one or more first co-receptor binding domain and one or more second co-receptor binding domain and one or more bridging binding domains. The binding domains may be directly fused or they may be connected via a linker, e.g., a polypeptide linker, including but not limited to any of those disclosed herein.

When the multicomponent polypeptide molecules comprise two or more polypeptides, the polypeptides may be linked via covalent bonds, such as, e.g., disulfide bonds, and/or noncovalent interactions. For example, heavy chains of human immunoglobulin IgG interact at the level of their CH3 domains directly, whereas, at the level of their CH2 domains, they interact via the carbohydrate attached to the asparagine (Asn) N84.4 in the DE turn. In particular embodiments, the multicomponent polypeptide molecules comprise one or more regions derived from an antibody or antigen-binding fragment thereof, e.g., antibody heavy chains or antibody light chains or fragments thereof. In certain embodiments, a surrogate polypeptide comprises two antibody heavy chain regions (e.g., hinge regions) bound together via one or more disulfide bond. In certain embodiments, a surrogate polypeptide comprises an antibody light chain region (e.g., a CL region) and an antibody heavy chain region (e.g., a CH1 region) bound together via one or more disulfide bond.

The multicomponent molecules, e.g., surrogate polypeptides, may be engineered to facilitate binding between two polypeptides. For example, Knob-into-holes amino acid modifications may be introduced into two different polypeptides to facilitate their binding. Knobs-into-holes amino acid (AA) changes is a rational design strategy developed in antibody engineering, used for heterodimerization of the heavy chains, in the production of bispecific IgG antibodies. AA changes are engineered in order to create a knob on the CH3 of the heavy chains from a first antibody and a hole on the CH3 of the heavy chains of a second antibody. The knob may be represented by a tyrosine (Y) that belongs to the ‘very large’ IMGT volume class of AA, whereas the hole may be represented by a threonine (T) that belongs to the ‘small’ IMGT volume class. Other means of introducing modifications into polypeptides to facilitate their binding are known and available in the art. For example, specific amino acids may be introduced and used for cross-linking, such as Cysteine to form an intermolecular disulfide bond.

Multicomponent molecules may have a variety of different structural formats, including but not limited to those as described in WO2019126398 and WO2020010308.

In one embodiment, a multicomponent molecule comprises an scFv or antigen-binding fragment thereof fused to a VHH or antigen-binding fragment thereof. In certain embodiments, the scFv specifically binds one or more first receptor, and the VHH specifically binds to one or more second receptor. In certain embodiments, the scFv specifically binds LRP5 and/or LRP6, and the VHH specifically binds one or more FZD receptor. In particular embodiments, the scFv or antigen-binding fragment thereof is fused directly to the VHH or antigen-binding fragment thereof, whereas in other embodiments, the two binding regions are fused via a linker moiety. In particular embodiments, the VHH is fused or linked to the N-terminus of the scFV, while in other embodiments, the VHH is fused to the C-terminus of the scFv.

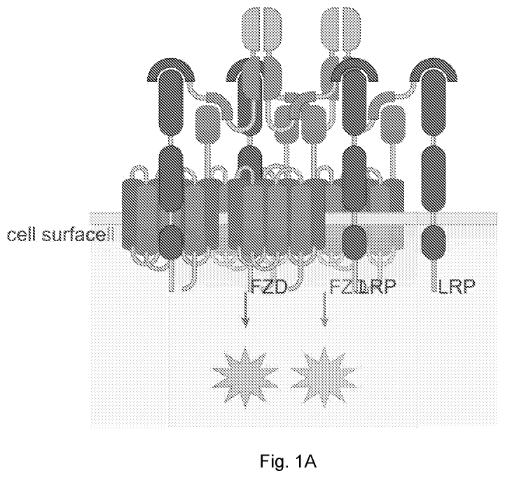

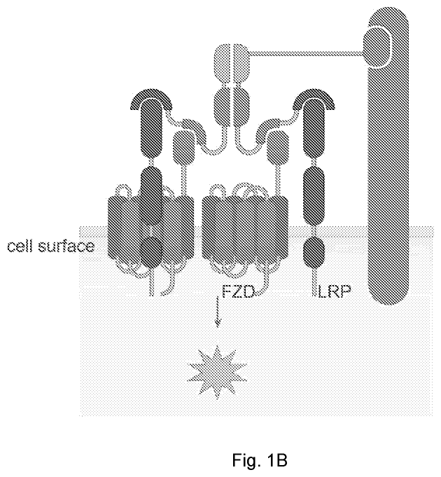

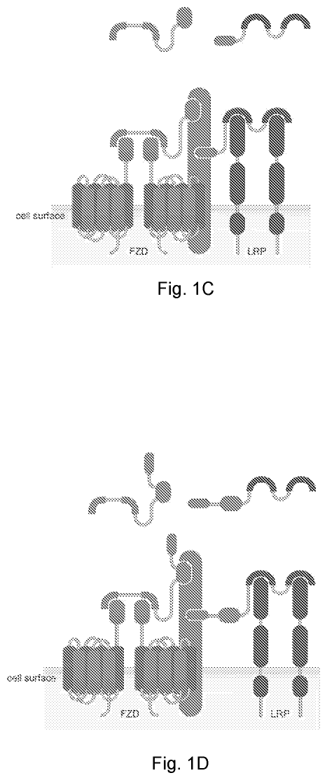

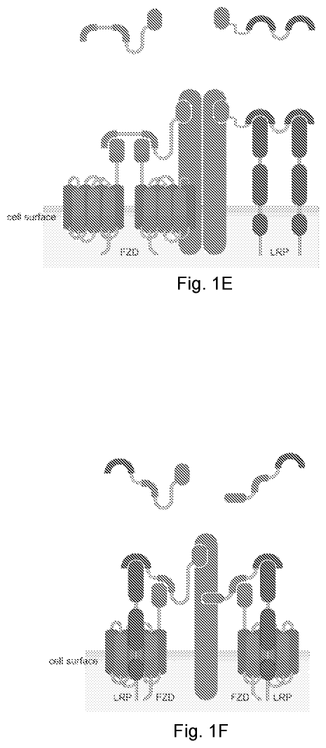

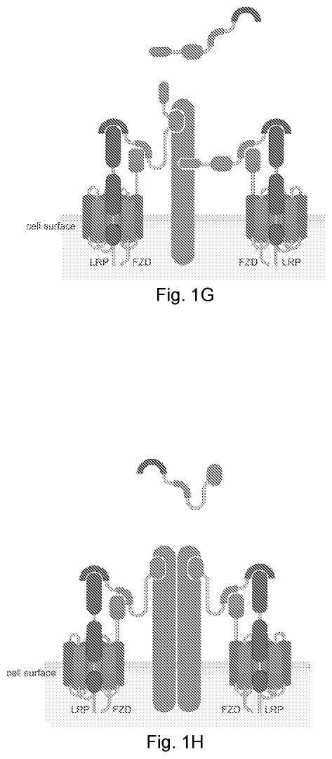

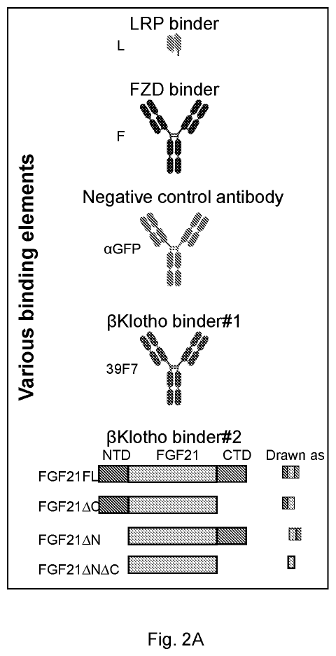

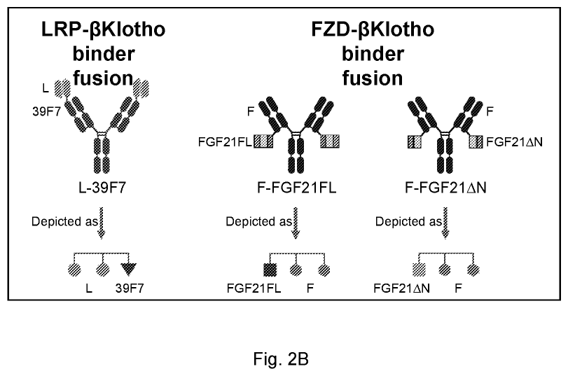

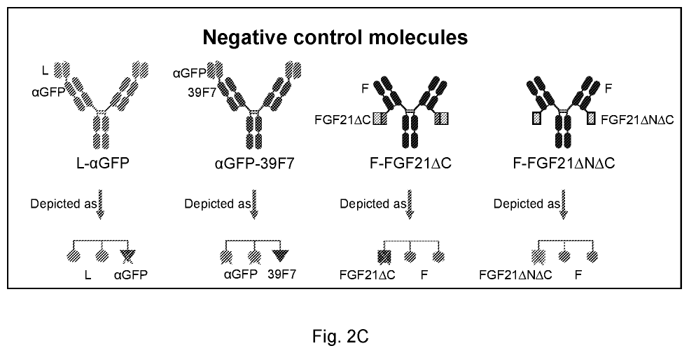

In various embodiments, including but not limited to those depicted in WO2019126398, WO2020010308, Table 2, Table 3, and FIGS. 1-3, a multicomponent polypeptide molecule comprises one or more Fab or antigen-binding fragment thereof and one or more V_{HH} or antigen-binding fragment thereof (or alternatively, one or more scFv or antigen-binding fragment thereof). In certain embodiments, the Fab specifically binds one or more FZD receptor, and the V_{HH} (or scFv) specifically binds LRP5 and/or LRP6. In certain embodiments, the Fab specifically binds LRP5 and/or LRP6, and the V_{HH} (or scFv) specifically binds one or more FZD receptor. In certain embodiments, the V_{HH} (or scFv) is fused to the N-terminus of the Fab, while in some embodiments, the V_{HH} (or scFv) is fused to the C-terminus of the Fab. In particular embodiments, the Fab is present in a full IgG format, and the V_{HH} (or scFv) is fused to the N-terminus and/or C-terminus of the IgG light chain. In particular embodiments, the Fab is present in a full IgG format, and the V_{HH} (or scFv) is fused to the N-terminus and/or C-terminus of the IgG heavy chain.

In particular embodiments, two or more VHHs (or scFvs) are fused to the IgG at any combination of these locations.

Fabs may be converted into a full IgG format that includes both the Fab and Fc fragments, for example, using genetic engineering to generate a fusion polypeptide comprising the Fab fused to an Fc region, i.e., the Fab is present in a full IgG format. The Fc region for the full IgG format may be derived from any of a variety of different Fcs, including but not limited to, a wild-type or modified IgG1, IgG2, IgG3, IgG4 or other isotype, e.g., wild-type or modified human IgG1, human IgG2, human IgG3, human IgG4, human IgG4Pro (comprising a mutation in core hinge region that prevents the formation of IgG4 half molecules), human IgA, human IgE, human IgM, or the modified IgG1 referred to as IgG1 LALAPG. The L235A, P329G (LALA-PG) variant has been shown to eliminate complement binding and fixation as well as Fc-γ dependent antibody-dependent cell-mediated cytotoxity (ADCC) in both murine IgG2a and human IgG1. These LALA-PG substitutions allow a more accurate translation of results generated with an “effectorless” antibody framework scaffold between mice and primates. In particular embodiments of any of the IgG disclosed herein, the IgG comprises one or more of the following amino acid substitutions: N297G, N297A, N297E, L234A, L235A, or P236G.

Non-limiting examples of bivalent and bispecific multicomponent polypeptide molecules of co-receptors that are bivalent towards both the one or more first receptor and one or more second receptor (e.g., FZD and LRP) are provided as the top four structures depicted in WO2019126398 and WO2020010308, where the VHH or scFv is depicted in white or grey, and the Fab or IgG is depicted in black. As shown, the VHH (or scFvs) may be fused to the N-termini of both light chains, to the N-termini of both heavy chains, to the C-termini of both light chains, or to the C-termini of both heavy chains. It is further contemplated, e.g., that VHH (or scFvs) could be fused to both the N-termini and C-termini of the heavy and/or light chains, to the N-termini of the light chains and the heavy chains, to the C-termini of the heavy and light chains, to the N-termini of the heavy chains and C-termini of the light chains, or to the C-termini of the heavy chains and the N-termini of the light chains. In other related embodiments, two or more VHH (or scFvs) may be fused together, optionally via a linker moiety, and fused to the Fab or IgG at one or more of these locations. In a related embodiment, the multicomponent polypeptide molecule has a Hetero-IgG format, whereas the Fab is present as a half antibody, and one or more VHH (or scFv) is fused to one or more of the N-terminus of the Fc, the N-terminus of the Fab, the C-terminus of the Fc, or the C-terminus of the Fab. A bispecific but monovalent to each receptor version of this format is depicted at FIG. 6. In certain embodiments, the Fab or antigen-binding fragment (or IgG) thereof is fused directly to the VHH (or scFv) or antigen-binding fragment thereof, whereas in other embodiments, the binding regions are fused via a linker moiety. In particular embodiments, the Fab is described herein or comprises any of the CDR sets described herein.

In various embodiments, including but not limited to those depicted in WO2019126398, WO2020010308, Table 2, Table 3, and FIGS. 1-3, an antigen binding molecule comprises one or more Fab or antigen-binding fragment thereof that binds one or more first receptor (e.g., FZD receptors) and one or more Fab or antigen-binding fragment thereof that binds to at least one or more second receptor (e.g., LRP5 and/or LRP6). In a particular embodiment, it comprises two Fab or antigen-binding fragments thereof that bind one or more first co-receptor and/or two Fab or antigen-binding fragments thereof that bind to one or more second co-receptor. In further embodiments, one or more of the Fab is present in a full IgG format, and in certain embodiments, both Fab are present in a full IgG format. In certain embodiments, the Fab in full IgG format specifically binds one or more first receptor (e.g., one or more FZD receptor), and the other Fab specifically binds at least one second receptor (e.g., LRP5 and/or LRP6). For example, the Fab specifically binds one or more FZD receptor, and the Fab in full IgG format specifically binds LRP5 and/or LRP6. In certain embodiments, the Fab specifically binds LRP5 and/or LRP6, and the Fab in full IgG format specifically binds one or more FZD receptor. In certain embodiments, the Fab is fused to the N-terminus of the IgG, e.g., to the heavy chain or light chain N-terminus, optionally via a linker. In certain embodiments, the Fab is fused to the N-terminus of the heavy chain of the IgG and not fused to the light chain. In particular embodiments, the two heavy chains can be fused together directly or via a linker. An example of such a bispecific and bivalent with respect to both receptors is shown in FIG. 1A. In other related embodiments, two or more VHHs may be fused together, optionally via a linker moiety, and fused to the Fab or IgG at one or more of these locations. In a related embodiment, the WNT multicomponent polypeptide molecule has a Hetero-IgG format, whereas one of the Fab is present as a half antibody, and the other Fab is fused to one or more of the N-terminus of the Fc, the N-terminus of the Fab, or the C-terminus of the Fc. A bispecific but monovalent to each receptor version of this format is depicted at FIG. 6. In certain embodiments, the Fab or antigen-binding fragment thereof is fused directly to the other Fab or IgG or antigen-binding fragment thereof, whereas in other embodiments, the binding regions are fused via a linker moiety. In particular embodiments, the one or both of the two Fabs are described herein or comprise any of the CDR sets described herein.

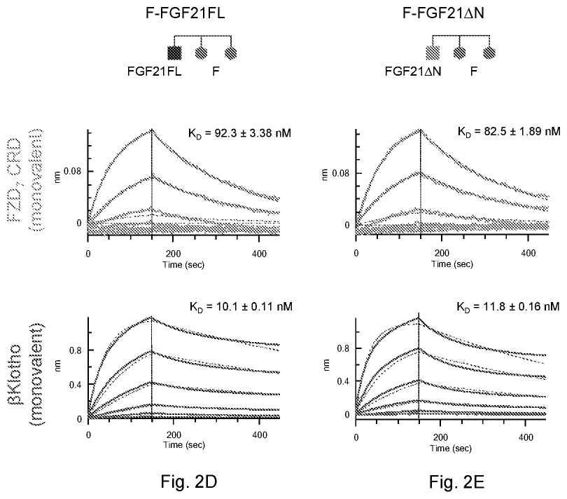

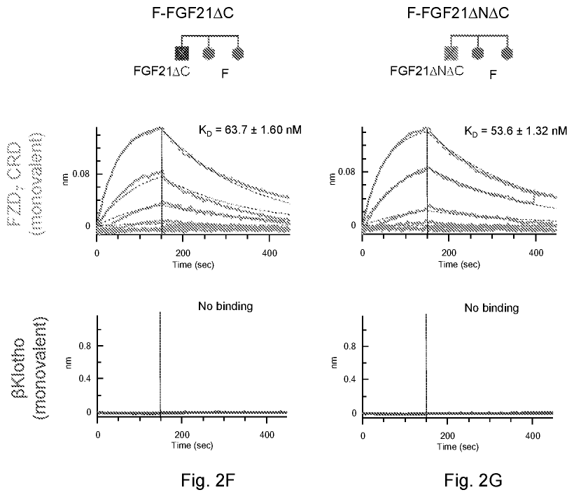

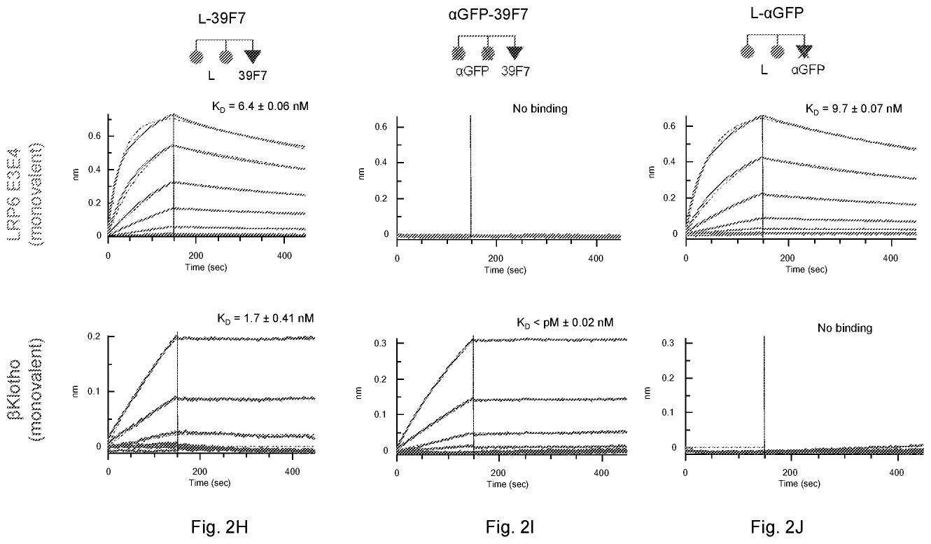

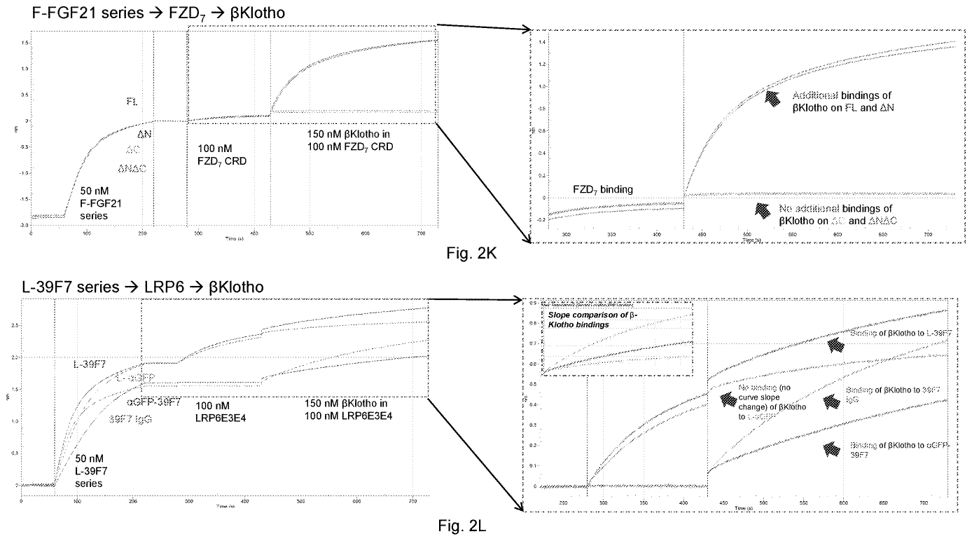

In certain embodiments, the antigen binding molecules have a format as described in PCT Application Publication No. WO2017/136820, e.g., a Fabs-in-tandem IgG (FIT-IG) format. Shiyong Gong, Fang Ren, Danqing Wu, Xuan Wu & Chengbin Wu (2017). FIT-IG also include the formats disclosed in, e.g., Gong, et al (2017) mAbs 9:118-1128. In certain embodiments, FIT-IGs combine the functions of two antibodies into one molecule by re-arranging the DNA sequences of two parental monoclonal antibodies into two or three constructs and co-expressing them in mammalian cells. Examples of FIT-IG formats and constructs are provided in FIGS. 1A and 1B and FIGS. 2A and 2B of PCT Application Publication No. WO2017/136820. In certain embodiments, FIT-IGs require no Fc mutation; no scFv elements; and no linker or peptide connector. The Fab-domains in each arm work “in tandem” forming a tetravalent bi-specific antibody with four active and independent antigen binding sites that retain the biological function of their parental antibodies In particular embodiments, WNT surrogates comprises a Fab and an IgG. In certain embodiments, the Fab binder LC is fused to the HC of the IgG, e.g., by a linker of various length in between. In various embodiment, the Fab binder HC can be fused or unfused to the LC of the IgG. A variation of this format has been called Fabs-in-tandem IgG (or FIT-Ig).

In certain embodiments, the WNT multicomponent polypeptide molecules have a format described in PCT Application Publication No. WO2009/080251 (Klein et al.), e.g., a CrossMab format. CrossMabs formats are also described in Schaefer et al. (2011) Proc. Natl. Acad. Sci USA 108:11187-11192. The CrossMab format allows correct assembly of two heavy chains and two light chains derived from existing antibodies to form a bispecific, bivalent IgG antibodies. The technology is based on the cross over the antibody domain within one Fab-arm of a bispecific IgG antibody in order to enable correct chain association. Various portions of the Fab can be exchanged, e.g., the entire Fab, the variable heavy and light chains, or the CH1-CL chains can be exchanged.

In further embodiments of the present invention, the FiT-Ig and CrossMab technologies are combined to create a multispecific, multivalent antigen binding molecule, Cross-FiT, as depicted in FIG. 1A and Table 2. Also contemplated is a linker between the crossed CL domain of the Fab and the Ig domains rather than between the CH1 and Ig domains. Additional antigen binding fragments, e.g., Fabs, VHH/sdAbs, and/or scFvs, can be appended to the Cross-FiT structure at various sites, e.g., the heavy or light chains and/or the C-terminus of the Fc domain to create multispecific antibodies.

In particular embodiments, multicomponent polypeptide molecules comprise two or more VHHs/sdAbs (or scFvs), including at least one that binds one or more first receptor and at least one that binds at least one second receptor. In certain embodiments, one of the binding regions is a VHH/sdAbs and the other is an scFv. Multicomponent polypeptide molecules comprising two or more VHH/sdAbs (or scFvs) may be formatted in a variety of configurations, including but not limited to those depicted in WO2019126398 and WO2020010308. In certain bispecific, bivalent formats, two or more VHH/sdAbs (or scFvs) are fused in tandem or fused to two different ends of an Fc, optionally via one or more linkers. Where linkers are present, the linker and its length may be the same or different between the VHH/sdAb (or scFv) and the other VHH/sdAb (or scFv), or between the VHH and Fc. For example, in certain embodiments, the VHH/sdAb is fused to the N-terminus, at either the heavy or light chain, and/or C-terminus of the IgG heavy chain. In particular embodiments, two or more VHH/sdAbs are fused to the IgG at any combination of these locations. In various embodiments, both VHH/sdAbs may be fused to the N-termini of the Fc, to the C-termini of the Fc, or one or more VHH/sdAb may be fused to either or both of an N-terminus or C-terminus of the Fc. In a related embodiment, the multicomponent polypeptide molecule has a Hetero-IgG format, whereas one VHH/sdAb is present as a half antibody, and the other is fused to the N-terminus of the Fc or the C-terminus of the Fc. In certain embodiments, the VHH/sdAb is fused directly to the other VHH/sdAb whereas in other embodiments, the binding regions are fused via a linker moiety. In particular embodiments, the VHH/sdAb are described herein or comprises any of the CDR sets described herein. In various embodiments, any of these formats may comprise one or more scFvs in place of one or more VHH/sdAbs.

In certain embodiments, a multicomponent polypeptide molecule is formatted as a diabody. The binders against the two co-receptors can also be linked together in a diabody (or DART) configuration. The diabody can also be in a single chain configuration. If the diabody is fused to an Fc, this will create a bivalent bispecific format. Without fusion to Fc, this would be a monovalent bispecific format. In certain embodiments, a diabody is a noncovalent dimer scFv fragment that consists of the heavy-chain variable (VH) and light-chain variable (VL) regions connected by a small peptide linker. Another form of diabody is a single-chain (Fv)2 in which two scFv fragments are covalently linked to each other.

As discussed, the multicomponent polypeptide molecules, in various embodiments, comprise one or more antibodies or antigen-binding fragments thereof disclosed herein. Thus, in particular embodiments, the surrogate comprises two polypeptides, wherein each polypeptide comprises an Nab or scFv that binds at least one first co-receptor and an Nab or scFv that binds at least one second co-receptor, optionally wherein one of the binding domains is an scFv and the other is an Nab. In certain embodiments, a surrogate comprises three polypeptides, wherein the first polypeptide comprises an antibody heavy chain and the second polypeptide comprises an antibody light chain, wherein the antibody heavy chain and light chain bind either receptor, and wherein the third polypeptide comprises a VHH/sdAb fused to a heavy chain Fc region or the light chain of the antibody, wherein the VHH/sdAb binds to either co-receptor. In other embodiments, the surrogates comprise four polypeptides, including two heavy chain polypeptides and two light chain polypeptides, wherein the two heavy chains and two light chains bind one or more first receptor, and further comprise one or more VHH/sdAb or scFv fused to one or more of the heavy chains and/or light chains, wherein the VHH/sdAb or scFv binds to one or more second co-receptor. In an illustrative embodiment, a WNT surrogate comprises at least four polypeptides, including two heavy chain polypeptides and two light chain polypeptides that bind either LRP5/6 or one or more FZDs, wherein the WNT surrogate further comprises a Fab that binds either LRP5/6 or one or more FZDs. For example, the Fab may comprise two polypeptides, each fused to one of the two heavy chain polypeptides, and two polypeptides, each fused to one of the two light chain polypeptides, or it may comprise two polypeptides each fused to one of the two heavy chain polypeptides and two additional polypeptides, each bound to one of the two polypeptides fused to the heavy chain polypeptides, thus making a second Fab. Other configurations disclosed herein may be used to produce different multicomponent polypeptide molecules.

Also contemplated are Ig molecules where the VL and VH domains of one Ig are appended with the VL and VH domains of a second antibody. This format is call Fv-Ig or 2Fv-Ig for a homodimer. The VL and VH domains from the second Ig are appended to the N-terminus of the VL and VH domains of the first Ig via short peptide linkers. This format preserves the natural antibody's avidity to cell surface receptors or to more than one receptor or co-receptor complexes (see, e.g., Wu, et al (2007) Nature Biotechnol. 25:1290-1297).

In certain embodiments, the antigen binding formats are multicomponent polypeptide molecules that comprise one or more polypeptides comprising two or more binding regions. For illustrative purposes, the two or more binding regions may be a first receptor binding regions or a second receptor binding regions, or they may comprise one or more first receptor binding region and one or more second receptor binding region. The binding regions may be directly joined or contiguous, or may be separated by a linker, e.g. a polypeptide linker, or a non-peptidic linker, etc. The length of the linker, and therefore the spacing between the binding domains can be used to modulate the signal strength, and can be selected depending on the desired use of the multicomponent polypeptide molecule. The enforced distance between binding domains can vary, but in certain embodiments may be less than about 100 angstroms, less than about 90 angstroms, less than about 80 angstroms, less than about 70 angstroms, less than about 60 angstroms, or less than about 50 angstroms. In some embodiments the linker is a rigid linker, in other embodiments the linker is a flexible linker. In certain embodiments where the linker is a peptide linker, it may be from about 1, 2, 3, 4, 5, 6, 7, 8, 9, 10, 11, 12, 13, 14, 15, 16, 17, 18, 19, 20 21, 22, 23, 24, 25, 26, 27, 28, 29, 30 or more amino acids in length, and is of sufficient length and amino acid composition to enforce the distance between binding domains. In some embodiments, the linker comprises or consists of one or more glycine and/or serine residues.

The multicomponent polypeptide molecule can be multimerized, e.g., through an Fc domain, by concatenation, coiled coils, polypeptide zippers, biotin/avidin or streptavidin multimerization, and the like. The multicomponent polypeptide molecules can also be joined to a moiety such as PEG, Fc, etc., as known in the art to enhance stability in vivo.

In certain embodiments, a multicomponent polypeptide molecule enhances or increases the co-receptors pathway signaling, e.g., in the case of WNT-β-catenin signaling, by at least 30%, 35%, 40%, 45%, 50%, 60%, 70%, 75%, 80%, 85%, 90%, 95%, 100%, 110%, 150%, 200%, 250%, 300%, 400% or 500%, as compared to the β-catenin signaling induced by a neutral substance or negative control as measured in an assay described above, for example as measured in the TOPFIash assay (see, e.g., Molinaar (1996) Cell 86:391-399). A negative control may be included in these assays. By way of example, WNT multicomponent polypeptide molecules may enhance β-catenin signaling by a factor of 2×, 5×, 10×, 100×, 1000×, 10000× or more as compared to the activity in the absence of the WNT multicomponent polypeptide molecule when measured, for example when measured in the TOPFIash assay.

In certain embodiments, functional properties of the multicomponent polypeptide molecules may be assessed using a variety of methods known to the skilled person, including e.g., affinity/binding assays (for example, surface plasmon resonance, competitive inhibition assays), cytotoxicity assays, cell viability assays, cell proliferation or differentiation assays in response to the native molecule/ligand, cancer cell and/or tumor growth inhibition using in vitro or in vivo models, including but not limited to any described herein. The multicomponent polypeptide molecules may also be tested for effects on one or both co-receptor internalization, in vitro and in vivo efficacy, etc. Such assays may be performed using well-established protocols known to the skilled person (see e.g., Current Protocols in Molecular Biology (Greene Publ. Assoc. Inc. & John Wiley & Sons, Inc., NY, NY); Current Protocols in Immunology (Edited by: John E. Coligan, Ada M. Kruisbeek, David H. Margulies, Ethan M. Shevach, Warren Strober 2001 John Wiley & Sons, NY, NY); or commercially available kits.

In certain embodiments, a binding region of a multicomponent polypeptide molecule (e.g., an antigen-binding fragment of an anti-FZD antibody) comprises one or more of the CDRs of the anti-co-receptor antibodies. In this regard, it has been shown in some cases that the transfer of only the VHCDR3 of an antibody can be performed while still retaining desired specific binding (Barbas et al., PNAS (1995) 92:2529-2533). See also, McLane et al., PNAS (1995) 92:5214-5218, Barbas et al., J. Am. Chem. Soc. (1994) 116:2161-2162).

Also disclosed herein is a method for obtaining an antibody or antigen binding domain specific for a co-receptor, the method comprising providing by way of addition, deletion, substitution or insertion of one or more amino acids in the amino acid sequence of a VH domain set out herein or a VH domain which is an amino acid sequence variant of the VH domain, optionally combining the VH domain thus provided with one or more VL domains, and testing the VH domain or VH/VL combination or combinations to identify a specific binding member or an antibody antigen binding domain specific for one or more co-receptors and optionally with one or more desired properties. The VL domains may have an amino acid sequence which is substantially as set out herein. An analogous method may be employed in which one or more sequence variants of a VL domain disclosed herein are combined with one or more VH domains.

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. See, generally, Davies et al. (1990) Annual Rev. Biochem. 59:439-473.

In certain embodiments, the multicomponent polypeptide molecules or binding regions thereof described herein have an affinity of less than about 10,000, less than about 1000, less than about 100, less than about 10, less than about 1 or less than about 0.1 nM, and in some embodiments, the antibodies may have even higher affinity for one or more co-receptors.

The constant regions of immunoglobulins show less sequence diversity than the variable regions, and are responsible for binding a number of natural proteins to elicit important biochemical events. In humans, there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region.

The Fc region of an antibody interacts with a number of Fc receptors and ligands, imparting an array of important functional capabilities referred to as effector functions. For IgG, the Fc region comprises Ig domains CH2 and CH3 and the N-terminal hinge leading into CH2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). All FcγRs bind the same region on Fc, at the N-terminal end of the Cg2 (CH2) domain and the preceding hinge. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273.) (pdb accession codes 1IIS and 1IIX) (Radaev et al., 2001, J Biol Chem 276:16469-16477.)

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical; however, FcγRIIIb does not have an intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Subjects with the V158 allotype respond favorably to rituximab treatment; however, subjects with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, they have at least one allele of the F158 FcγRIIIa.

The Fc region is also involved in activation of the complement cascade. In the classical complement pathway, C1 binds with its C1q subunits to Fc fragments of IgG or IgM, which has formed a complex with antigen(s). In certain embodiments of the invention, modifications to the Fc region comprise modifications that alter (either enhance or decrease) the ability of a FZD-specific antibody as described herein to activate the complement system (see e.g., U.S. Pat. No. 7,740,847). To assess complement activation, a complement-dependent cytotoxicity (CDC) assay may be performed (See, e.g., Gazzano-Santoro et al., J. Immunol. Methods, 202:163 (1996).

Thus in certain embodiments, the present invention provides the multicomponent polypeptide molecules having a modified Fc region with altered functional properties, such as reduced or enhanced CDC, ADCC, or ADCP activity, or enhanced binding affinity for a specific FcγR or increased serum half-life. Other modified Fc regions contemplated herein are described, for example, in issued U.S. Pat. Nos. 7,317,091; 7,657,380; 7,662,925; 6,538,124; 6,528,624; 7,297,775; 7,364,731; Published U.S. Applications US2009092599; US20080131435; US20080138344; and published International Applications WO2006/105338; WO2004/063351; WO2006/088494; WO2007/024249.

Structurally, the Fc region can be important for proper assembly of the msAb. In particular, modifications to the CH3 domain such as knobs-in-hole (see, e.g., WO1996/027011; and WO1998/050431) or Azymetric mutations (see, e.g., WO2012/58768) can prevent heavy chain mispairing. The present invention utilizes these mutations in certain Fc embodiments.

The multicomponent molecules disclosed herein may also be modified to include an epitope tag or label, e.g., for use in purification or diagnostic applications. There are many linking groups known in the art for making antibody conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

In certain embodiments, and antigen-binding fragments thereof against one co-receptor and/or antibodies and antigen-binding fragments thereof against the other co-receptor present within a multicomponent polypeptide molecule are monoclonal. In certain embodiments, they are humanized.

The present invention further provides in certain embodiments an isolated nucleic acid encoding a polypeptide present in a multicomponent polypeptide molecule. Nucleic acids include DNA and RNA. These and related embodiments may include polynucleotides encoding antibody fragments that bind one or more co-receptors. The term “isolated polynucleotide” as used herein shall mean a polynucleotide of genomic, cDNA, or synthetic origin, or some combination thereof, which by virtue of its origin, the isolated polynucleotide: (1) is not associated with all or a portion of a polynucleotide in which the isolated polynucleotide is found in nature; (2) is linked to a polynucleotide to which it is not linked in nature, or (3) does not occur in nature as part of a larger sequence. An isolated polynucleotide may include naturally occurring and/or artificial sequences.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

It will be appreciated by those of ordinary skill in the art that, as a result of the degeneracy of the genetic code, there are many nucleotide sequences that encodes an antibody as described herein. Some of these polynucleotides bear minimal sequence identity to the nucleotide sequence of the native or original polynucleotide sequence encoding a polypeptide within a multicomponent polypeptide molecule. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the polypeptides described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode a polypeptide present in a multicomponent polypeptide molecule, to alter one or more properties of the encoded polypeptide, such as the binding affinity, or the function of a particular Fc region, or the affinity of the Fc region for a particular FcγR. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are readily commercially-available and their use is generally well-known to those skilled in the art. Double-stranded plasmids are also routinely employed in site directed mutagenesis that eliminates the step of transferring the gene of interest from a plasmid to a phage.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

In many embodiments, one or more nucleic acids encoding a polypeptide of multicomponent WNT molecule are introduced directly into a host cell, and the cell incubated under conditions sufficient to induce expression of the encoded polypeptides. The surrogate polypeptides of this disclosure may be prepared using standard techniques well known to those of skill in the art in combination with the polypeptide and nucleic acid sequences provided herein. The polypeptide sequences may be used to determine appropriate nucleic acid sequences encoding the particular polypeptide disclosed thereby. The nucleic acid sequence may be optimized to reflect particular codon “preferences” for various expression systems according to standard methods well known to those of skill in the art.

According to certain related embodiments there is provided a recombinant host cell which comprises one or more constructs as described herein, e.g., a vector comprising a nucleic acid encoding a multicomponent polypeptide molecule or polypeptide thereof; and a method of production of the encoded product, which method comprises expression from encoding nucleic acid therefor. Expression may conveniently be achieved by culturing under appropriate conditions recombinant host cells containing the nucleic acid. Following production by expression, an antibody or antigen-binding fragment thereof, may be isolated and/or purified using any suitable technique, and then used as desired.

Polypeptides, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of polypeptides, e.g., antibodies and antigen-binding fragments thereof, in prokaryotic cells such as E. coli is well established in the art. For a review, see for example Pluckthun, A. Bio/Technology 9:545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4:573-576; Trill J. J. et al. (1995) Curr. Opinion Biotech 6:553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The present invention also provides, in certain embodiments, a method which comprises using a construct as stated above in an expression system in order to express a particular polypeptide such as a WNT chimeric activator molecule. The term “transduction” is used to refer to the transfer of genes from one bacterium to another, usually by a phage.

Amino acid sequence modification(s) of any of the polypeptides described herein are contemplated. For example, it may be desirable to improve the binding affinity and/or other biological properties of the multicomponent polypeptide molecule. For example, amino acid sequence variants of a multicomponent polypeptide molecule may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final multicomponent polypeptide molecule, provided that the final construct possesses the desired characteristics (e.g., high affinity binding to one or more co-receptors). The amino acid changes also may alter post-translational processes of the antibody, such as changing the number or position of glycosylation sites. Any of the variations and modifications described above for polypeptides of the present invention may be included in antibodies of the present invention.

The present disclosure provides variants of any of the polypeptides (e.g., multicomponent molecule, multicomponent polypeptide molecules, or antibodies or antigen-binding fragments thereof) disclosed herein. In certain embodiments, a variant has at least 90%, at least 95%, at least 98%, or at least 99% identity to a polypeptide disclosed herein. In certain embodiments, such variant polypeptides bind to one or more first co-receptors, and/or to one or more second co-receptors, at least about 50%, at least about 70%, and in certain embodiments, at least about 90% as well as a t multicomponent polypeptide molecule specifically set forth herein. In further embodiments, such variant multicomponent polypeptide molecules bind to one or more first co-receptor, and/or to one or more second co-receptor, with greater affinity than the multicomponent polypeptide molecules set forth herein, for example, that bind quantitatively at least about 105%, 106%, 107%, 108%, 109%, or 110% as well as an antibody sequence specifically set forth herein.

In particular embodiments, the multicomponent polypeptide molecule or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and/or b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target. In a further embodiment, the antibody, or antigen-binding fragment thereof, is a variant antibody or antigen-binding fragment thereof wherein the variant comprises a heavy and light chain identical to the selected antibody except for up to 8, 9, 10, 11, 12, 13, 14, 15, or more total amino acid substitutions in the CDR regions of the VH and VL regions. In this regard, there may be 1, 2, 3, 4, 5, 6, 7, 8, or in certain embodiments, 9, 10, 11, 12, 13, 14, 15 more total amino acid substitutions in the (collective) CDR regions of the selected antibody. Substitutions may be in CDRs either in the VH and/or the VL regions. (See e.g., Muller, 1998, Structure 6:1153-1167).

In particular embodiments, the multicomponent polypeptide molecule or a binding region thereof, e.g., a Fab, scFv, or VHH/sdAb, may have: a) a heavy chain variable region having an amino acid sequence that is at least 80% identical, at least 95% identical, at least 90%, at least 95% or at least 98% or 99% identical, to the heavy chain variable region of an antibody or antigen-binding fragments thereof described herein; and/or b) a light chain variable region having an amino acid sequence that is at least 80% identical, at least 85%, at least 90%, at least 95% or at least 98% or 99% identical, to the light chain variable region of an antibody or antigen-binding fragments thereof described herein.

A polypeptide has a certain percent “sequence identity” to another polypeptide, meaning that, when aligned, that percentage of amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

Of interest is the BestFit program using the local homology algorithm of Smith and Waterman (Advances in Applied Mathematics 2:482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

In particular embodiments, the multicomponent polypeptide molecule or a binding region thereof, e.g., a Fab, scFv, or VHH may comprise: a) a heavy chain variable region comprising: i. a CDR1 region that is identical in amino acid sequence to the heavy chain CDR1 region of a selected antibody described herein; ii. a CDR2 region that is identical in amino acid sequence to the heavy chain CDR2 region of the selected antibody; and iii. a CDR3 region that is identical in amino acid sequence to the heavy chain CDR3 region of the selected antibody; and b) a light chain variable domain comprising: i. a CDR1 region that is identical in amino acid sequence to the light chain CDR1 region of the selected antibody; ii. a CDR2 region that is identical in amino acid sequence to the light chain CDR2 region of the selected antibody; and iii. a CDR3 region that is identical in amino acid sequence to the light chain CDR3 region of the selected antibody; wherein the antibody specifically binds a selected target (e.g., a FZD receptor, such as FZD1). In a further embodiment, the antibody, or antigen-binding fragment thereof, is a variant antibody wherein the variant comprises a heavy and light chain identical to the selected antibody except for up to 8, 9, 10, 11, 12, 13, 14, 15, or more amino acid substitutions in the CDR regions of the VH and VL regions. In this regard, there may be 1, 2, 3, 4, 5, 6, 7, 8, or in certain embodiments, 9, 10, 11, 12, 13, 14, 15 more amino acid substitutions in the CDR regions of the selected antibody. Substitutions may be in CDRs either in the VH and/or the VL regions. (See e.g., Muller, 1998, Structure 6:1153-1167).

Determination of the three-dimensional structures of representative polypeptides (e.g., variant FZD binding regions or LRP5/6 binding regions and bridging regions of WNT multicomponent polypeptide molecules as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309:1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of binding regions include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/. Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106:765), which assess force field calculations, and analysis (see also, Eisenfield et al. (1991) Am. J. Physiol. 261: C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

### IV. WNT SIGNAL ENHANCING MOLECULES (WNT ENHANCERS)

In an embodiment, a multicomponent WNT molecule, as described herein, possesses at least one FZD binding domain, at least one LRP binding domain, and at least one bridging molecule; the WNT multicomponent polypeptide molecule modulates WNT signaling. In particular embodiments, the binding domains bind to their target when the target is present on the cell surface, e.g., they may bind to an epitope within the extracellular domain of their target.

In certain embodiments, the multicomponent WNT molecule, described herein, possesses two FZD binding domains tethered to the bridging molecule by a binding domain specific for a first epitope on the bridging molecule; two LRP binding domains tethered to the bridging molecule by a binding domain specific for a second epitope at least one different antigen binding domain molecule; and, the tethering of the FZD and LRP binding domains to the bridging molecule forms a WNT mimetic capable of activating WNT signaling.

In certain embodiments, the multicomponent WNT molecule, described herein, possesses the binding domain specific for a first epitope of the bridging molecule and the FZD binding domain are joined directly or by a linker, the binding domain specific for a second epitope of the bridging molecule and the LRP binding domain are joined directly, or by a linker, and the binding domain of the first epitope and second epitope of the bridging molecule are identical or different.

In another embodiment, the multicomponent WNT molecule, reported herein, possess at least one FZD binding domain linked to one LRP binding domain which is tethered to at least one different antigen binding domain by a binding domain specific for a first epitope on the bridging molecule, at least one FZD binding domain linked to one LRP binding domain which is tethered to at least one different antigen binding domain by a binding domain specific for a second epitope on the bridging molecule, and the tethering of the FZD and LRP binding domains to the bridging receptor forms a WNT mimetic capable of activating WNT signaling.

In certain embodiments, the multicomponent WNT molecule, described herein, possesses the binding domain specific for a first epitope of the bridging molecule and the FZD binding domain are joined directly or by a linker, and the binding domain specific for a second epitope of the bridging molecule and the LRP binding domain are joined directly, or by a linker, wherein the binding domain of the first epitope and second epitope of the bridging molecule are identical or different, and could monomeric or multimeric.

### V. TARGETING MOLECULES

Specific cell types and cells within specific tissue may comprise one or more cell- or tissue-specific surface molecule, such as a cell surface receptor. As used herein, the molecule is said to be cell- or tissue-specific if a greater amount of the molecule is present on the specific cell or tissue type as compared to one or more other cell or tissue types, or any other cell or tissue type. In certain embodiments, the greater amount is at least two-fold, at least five-fold, at least 10-fold, at least 20-fold, at least 50-fold, or at least 100-fold as compared to the amount in the one or more other cell or tissue types, or any other cell or tissue type. In particular embodiments, the cell-specific surface molecule has increased or enhanced expression on a target organ, tissue or cell type, e.g., an organ, tissue or cell type in which it is desirous to enhance WNT signaling, e.g., to treat or prevent a disease or disorder, e.g., as compared to one or more other non-targeted organs, tissues or cell types. In certain embodiments, the cell-specific surface molecule is preferentially expressed on the surface of the target organ, tissue or cell type as compared to one or more other organ, tissue or cell types, respectively. For example, in particular embodiments, a cell surface receptor is considered to be a tissue-specific or cell-specific cell surface molecule if it is expressed at levels at least two-fold, at least five-fold, at least 10-fold, at least 20-fold, at least 30-fold, at least 40-fold, at least 50-fold, at least 100-fold, at least 500-fold, or at least 1000-fold higher in the target organ, tissue or cell than it is expressed in one or more, five or more, all other organs, tissues or cells, or an average of all other organs, tissue or cells, respectively. In certain embodiments, the tissue-specific or cell-specific cell surface molecule is a cell surface receptor, e.g., a polypeptide receptor comprising a region located within the cell surface membrane and an extracellular region to which the targeting module can bind. In various embodiments, the methods described herein may be practiced by specifically targeting cell surface molecules that are only expressed on the target tissue or a subset of tissues including the target tissue, or by specifically targeting cell surface molecules that have higher levels of expression on the target tissue as compared to all, most, or a substantial number of other tissues, e.g., higher expression on the target tissue than on at least two, at least five, at least ten, or at least twenty other tissues.

The targeted tissue may be bound by a targeting module, e.g., a binding domain that specifically binds to the tissue specific receptor. The targeted tissue may be any tissue, e.g., any mammalian tissue or cell type. In certain embodiments, the targeted tissue may be present in any organ. In certain embodiments, the target tissue is bone tissue, liver tissue, skin tissue, stomach tissue, intestine tissue, oral mucosa tissue, kidney tissue, central nervous system tissue, mammary gland tissue, taste bud tissue, ovary tissue, inner ear tissue (including cochlear and vestibular tissues), hair follicles, pancreas tissue, retina tissue, cornea tissue, heart tissue or lung tissue, and the targeting module binds to a tissue-specific cell surface molecule (e.g., a cell surface receptor) preferentially expressed on bone tissue, liver tissue, skin tissue, stomach tissue, intestine tissue, oral mucosa tissue, kidney tissue, central nervous system tissue, mammary gland tissue, taste bud tissue, ovary tissue, inner ear tissue (including cochlear and vestibular tissues), hair follicles, pancreas tissue, retina tissue, cornea tissue, heart tissue or lung tissue, respectively.

The targeting module may bind to any cell type, e.g., any cell within any tissue, organ or animal, including but not limited to mammals, such as humans. In certain embodiments, the tissue-specific WNT surrogate-signal enhancing combination molecule binds to specific cell types, e.g., specific cell types associated with a target tissue. For example, in liver tissue, the targeting module may bind to hepatocytes, precursors and stem cells of hepatocytes, biliary tract cells, and/or endothelial or other vascular cells. Examples of liver specific targeting molecules are provided, e.g., in WO 2018/140821 and WO 2020/014271, both of which are incorporated by reference herein. For example, in bone tissue, the targeting module may bind osteoblasts, precursors of osteoblasts, mesenchymal stem cells, stem cells and precursor cells that give rise to bone, cartilage and/or other cells present in bone tissue. Cell types present in various tissues, including but not limited to the tissues described herein, are known in the art, and in various embodiments, the tissue specific WNT signal enhancing molecules described herein may bind any of them.

### VI. LINKERS

In certain embodiments, the multicomponent targeting molecule, for example FZD molecule, LRP molecule, and the bridging molecule, are bound or fused directly to each other, whereas in other embodiments, they are separated by a linker, e.g., a polypeptide linker, or a non-peptidyl linker, etc. In particular embodiments, a linker is an Fc linker, e.g., a region of an antibody Fc domain capable of dimerizing with another Fc linker, e.g., via one or more disulfide bonds. In another particular embodiment, a linker is albumin, e.g., human serum albumin, where the targeting and action modules are on the N- and C-termini of albumin.

In certain embodiments, particularly when joining two polypeptides, the linker is made up of amino acids linked together by peptide bonds. In particular embodiments, the linker comprises, in length, from 1 up to about 40 amino acid residues, from 1 up to about 20 amino acid residues, or from 1 to about 10 amino acid residues. In certain embodiments, the amino acid residues in the linker are from among the twenty canonical amino acids, and in certain embodiments, selected from cysteine, glycine, alanine, proline, asparagine, glutamine, and/or serine. In certain embodiments, a linker comprises one or more non-natural amino acids. In some embodiments, a peptidyl linker is made up of a majority of amino acids that are sterically unhindered, such as glycine, serine, and alanine linked by a peptide bond. Certain linkers include polyglycines, polyserines, and polyalanines, or combinations of any of these. Some exemplary peptidyl linkers are poly(Gly)1-8, particularly (Gly)3, (Gly)4 (SEQ ID NO: 14), (Gly)5 (SEQ ID NO: 15), (Gly)6 (SEQ ID NO: 16), (Gly)7 (SEQ ID NO: 17), and (Gly)8 (SEQ ID NO: 18) as well as, poly(Gly)4 Ser (SEQ ID NO: 19), poly(Gly-Ala)2 (SEQ ID NO: 20), poly(Gly-Ala)3 (SEQ ID NO: 21), poly(Gly-Ala)4 (SEQ ID NO: 22) and poly(Ala)1-8 (SEQ ID NO: 23-27). Other specific examples of peptidyl linkers include (Gly)5Lys (SEQ ID NO: 28), and (Gly)5LysArg (SEQ ID NO: 29). To explain the above nomenclature, for example, (Gly)3Lys(Gly)4 means Gly-Gly-Gly-Lys-Gly-Gly-Gly-Gly (SEQ ID NO: 30). Other combinations of Gly and Ala are also useful. Additionally, a peptidyl linker can also comprise a non-peptidyl segment such as a 6 carbon aliphatic molecule of the formula —CH2-CH2-CH2-CH2-CH2-CH2-. The peptidyl linkers can be altered to form derivatives as described herein.

Illustrative non-peptidyl linkers include, for example, alkyl linkers such as —NH—(CH2) s-C(O)—, wherein s=2-20. These alkyl linkers may further be substituted by any non-sterically hindering group such as lower alkyl (e.g., C1-C6) lower acyl, halogen (e.g., Cl, Br), CN, NH2, phenyl, etc. Non-peptide portions of the inventive composition of matter, such as non-peptidyl linkers or non-peptide half-life extending moieties can be synthesized by conventional organic chemistry reactions. Chemical groups that find use in linking binding domains include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hydrazone, lipids, and the like, as known in the art.

The linkage between domains may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O) n where n is from 1 to about 12; (CH₂CH₂NH) n, where n is from 1 to about 12; [(CH₂)n(C═O)NH(CH₂)ₘ]_{z}, where n and m are from 1 to about 6, and z is from 1 to about 10; [(CH₂)nOPO₃(CH₂)ₘ]_{z} where n and m are from 1 to about 6, and z is from 1 to about 10. Such linkers may include polyethylene glycol, which may be linear or branched.

In certain embodiments, the domains may be joined through a homo- or heterobifunctional linker. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio) propionic acid N-hydroxysuccinimide ester (SPDP); N,N′-(1,3-phenylene) bismaleimide; N,N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl) butyrate (SMPB), an extended chain analog of MBS. In certain embodiments, the succinimidyl group of these cross-linkers reacts with a primary amine, and the thiol-reactive maleimide forms a covalent bond with the thiol of a cysteine residue.

Other reagents useful include: homobifunctional cross-linking reagents including bismaleimidohexane (“BMH”); p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like.

### VII. CELL TARGETING ACTIVATORS VIA BRIDGING RECEPTOR(S)

The present invention encompasses a novel concept to achieve cell targeting for ligand/receptor systems that involves multicomponent receptor complexes, where a productive signaling competent receptor complex formation is mediated through a “bridging element”, using “chimeric activator” approach based on the cooperativity concept. When an “targeting element” is tethered to the mutant “activity element”, the “targeting element” helps to increase the local concentration of the mutant “activity element” on the desired target cell surface, driving engagement of signaling receptor and subsequent intracellular signaling activation, effectively left shift the activity dose response curve (increase potency) on target cell and create the selectivity between target vs non-target cells.

The concept of cell targeting for ligand/receptor systems that involves multicomponent receptor complexes, where a productive signaling competent receptor complex formation is mediated through a “bridging element” is tested using the WNT/β-catenin signaling pathway as a model system.

WNT pathway is highly conserved across species and crucial for embryonic development, and adult tissue homeostasis and regeneration (Nusse and Clevers, 2017). WNT induced signaling through β-catenin stabilization has been widely studied and is achieved by ligand binding to frizzled (FZD) and low-density lipoprotein receptor-related protein (LRP) family of receptors. There are nineteen mammalian WNTs, 10 FZDs (FZD1-10), and 2 LRPs (LRP5 and LRP6). WNTs are highly hydrophobic due to lipidation that is required for function and are promiscuous, capable of binding and activating multiple FZD and LRP pairs (Janda et al., 2012, Kadowaki et al., 1996, Dijksterhuis et al., 2015). Elucidating the functions of individual FZDs in tissues has been hampered by difficulties in producing the ligands and lack of receptor and tissue selectivity. Recent breakthroughs in the development of WNT-mimetic molecules have largely resolved the production and receptor specificity challenges (Janda et al., 2017, Chen et al., 2020, Tao et al., 2019, Miao et al., 2020). While tissue selectivity could be partly achieved by tissue injury as damaged tissues seem more sensitive to WNTs (Xie et al., 2022), it would still represent a significant technical advancement to be able to target WNTs to specific cells and tissues.

The WNT mimetics reported so far are all bispecific and can simultaneously bind to FZDs and LRPs, and the optimal stoichiometry (at least for the antibody based molecules) are tetravalent bispecific (2:2 format), requiring two FZD binders and two LRP binders in the same molecule to achieve efficient signaling (Tao et al., 2019, Chen et al., 2020). We took advantage of the fact that two FZD binders alone, two LRP binders alone, or a molecule with one FZD binder and one LRP binder (1:1 format) do not signal. Cell specificity could be achieved by attaching an “targeting element” (capable of binding to another cell surface receptor, called a bridging receptor or bridging molecule here) to these three inactive molecules, and signaling competent receptor complexes consisting of two FZDs and two LRPs could then be assemble via the bridging receptor on the target cell surface. This approach reduces or eliminates the need to mutate and reduce the affinity of the “active elements” toward signaling receptors, and creates a highly cell specific activation of the signaling pathway as the individual components are inactive.

FIG. 1A. shows one optimized design for a WNT mimetic that is a tetravalent bispecific antibody-based molecule. It is important to highlight that efficient WNT/β-catenin signaling requires two FZD binding domains and two LRP binding domains in one molecule (Chen et al., 2020). FIG. 1B shows a cell targeted WNT mimetic based on the “chimeric activator” concept where an “targeting element” is tethered to the WNT mimetic. These molecules are representative illustrations, orientations and attachment locations of the different elements can be varied. The adjustments of the affinities of the different binding components can influence the separation between targeting vs non-targeting cells.

FIGS. 1C-1E show the cell targeting approach using the bridging receptor concept. The first step of this concept is to split the active molecule into inactive components. Using the tetravalent bispecific WNT mimetic as an example, among many different ways of splitting this molecule, we explored two ways for illustration purpose. The first method is to split the tetravalent WNT mimetic into one molecule having two FZD binding arms (2:0) and a second molecule having two LRP binding arms (0:2) (FIG. 1C-1E). These two inactive components can be assembled by a cell specific bridging receptor in several ways. If the bridging receptor exists as a monomer, as depicted in FIG. 1C, each separate molecule could be tethered to a “bridging element” binding to a different epitope on the bridging receptor, creating a final receptor complex on the cell surface consisting of two FZDs and two LRPs that would be competent to trigger signaling. The two different “bridging elements” could also be tethered to the two separate FZD and LRP binders as depicted in FIG. 1D for the assembly of the active receptor complex on the cell surface. If the bridging receptor exists as a multimeric complex of its own, a single “bridging element” could be tethered to the two different FZD and LRP molecules as depicted in FIG. 1E.

FIGS. 1F-1H show the method to split the tetravalent WNT mimetic is to split into two identical 1:1 molecules that consists of one FZD binding arm and one LRP binding arm. Similar to the scenario in FIG. 1C, if the bridging receptor exists as a monomer, two “bridging elements” binding to two different epitopes on the bridging receptor could be tethered to two separate 1:1 “activity element”, which can then assemble via bridging receptor to create a competent productive signaling complex (FIG. 1F). The two different “bridging elements” could also be tethered together to the one 1:1 “activity element” (FIG. 1G). Unlike the scenarios shown in FIG. 1C-IF which employs two distinct “activity elements” for signaling, in the scenario in FIG. 1G, only a single molecule is needed to assemble a productive receptor signaling complex with cell targeting capability. If the bridging receptor exist as a multimeric complex of its own, a single “bridging element” could be tethered to a single “activity element” to create one molecule that achieve cell targeting (FIG. 1H). Additional variations of how the elements can be combined could be envisioned under the bridging receptor concept.

Tandem scFv multicomponent polypeptide are generated and assembled by linking or directly fusing a first scFv to either the C- or N-terminus of a second scFv molecule. In one format, the first scFv can bind to one or more FZD receptors and the second scFv can bind to one or more LRP receptors. In an alternative format, the first scFv can bind to one or more LRP receptors, and the second scFv can bind to one or more FZD receptors. One of the scFv molecules can also be linked or directly fused at its C-terminus to the N-terminus of an Fc molecule. In certain embodiments the WNT enhancer is linked or fused to the N-terminus of a first scFv, which in turn is linked or fused to the N-terminus of the second scFv, which is linked or fused to the N-terminus of the Fc molecule. In alternative embodiments, the WNT enhancer is linked or fused to the C-terminus of the Fc molecule, which in turn is linked or fused to the C-terminus of one scFv molecule, which is linked or fused at its N-terminus to the C-terminus of a second scFv molecule.

Fab-IgG molecules, where the FZD and LRP binders are Fabs can be assembled in various approaches, such as charge pairing, knobs-in-holes, crossover of heavy and light chains of the Fabs, etc. In charge pairing the heavy chain (VH-CH1) domain of an anti-LRP6 Fab or an anti-FZD Fab, through direct fusion or a linker of 5, 10, or 15-mer amino acids, are fused in tandem with the N-terminus of the heavy chain (VH-CH1-CH2-CH3) of an anti-FZD or anti-LRP binder. In certain embodiments, also known as Fabs-in tandem (FiT), both VH-CH1 domains of anti-LRP6 and anti-FZD contain three amino acid mutations (Q39D, Q105D, S183K in the anti-LRP6 Fab; Q39K, Q105K, S183E in anti-FZD Fab) each for proper paring with their own partner light chains, which also contain three complementary amino acid mutations (Q38K, A/S43K, S176E in anti-LRP6 light chain; Q38D, A/S43D, S176K in the anti-FZD light chain). The order of the anti-LRP6 and anti-FZD Fabs could be reversed, where the anti-FZD binder is a Fab and is fused to anti-LRP binder which is in IgG format. In certain embodiments, the WNT enhancer can be attached to the Fab to the N-terminus of either the Vh or VI the Fab furthest from the IgG domain. In other embodiments, the WNT enhancer is attached to C-terminus of the IgG domain.

HC-LC cross over approach for Fab-on-IgG format: The light chain (VL-CL) domains of anti-LRP6 binder is, through direct fusion or a linker of 5, 10, or 15-mer amino acids, fused in tandem with the N-terminus of the heavy chain (VH-CH1-CH2-CH3) of an anti-FZD binder. The second construct was VH-CH1 of the anti-LRP6 binder and the third construct was VL-CL of the anti-FZD binder. Similar to the example above, the order of the anti-LRP6 and the anti-FZD binders could be reversed, where anti-FZD binder Fab is fused to the N-terminus of the anti-LRP binder which is in IgG format. Also as above the WNT enhancer can be attached to N-terminus of the VH or VL of the crossover Fab furthest from the IgG domain, or attached to the C-terminus of the IgG domain.

In certain embodiments, the molecules comprise one or more polypeptide(s) comprising or consisting of a sequence selected from any of SEQ ID NOs: 1-13, or a functional fragment or variant thereof. In particular embodiments, a variant comprises at least 80%, at least 85%, at least 90%, at least 95%, at least 98%, or at least 99% identity to any one of SEQ ID NOs: 1-13. In certain embodiments, a fragment comprises at least 50%, at least 60%, at least 70%, at least 80%, at least 90%, at least 95%, or at least 98% of a contiguous sequence of any of SEQ ID NOs: 1-13 or a variant of any of SEQ ID NOs: 1-13. In certain embodiments, a functional fragment comprises an FGF21 sequence, an F12578 sequence, and/or a L sequence present in any of SEQ ID NOs: 1-13, or a variant thereof.

### VIII. COMPOSITIONS

Pharmaceutical compositions comprising a multicomponent polypeptide molecule described herein and one or more pharmaceutically acceptable diluent, carrier, or excipient are also disclosed.

In further embodiments, pharmaceutical compositions comprising a polynucleotide comprising a nucleic acid sequence encoding a multicomponent polypeptide molecule described herein and one or more pharmaceutically acceptable diluent, carrier, or excipient are also disclosed. In particular embodiments, the pharmaceutical composition further comprises one or more polynucleotides comprising a nucleic acid sequence encoding a naturally occurring co-receptor ligand polypeptide. In certain embodiments, the polynucleotides are DNA or mRNA, e.g., a modified mRNA. In particular embodiments, the polynucleotides are modified mRNAs further comprising a 5′ cap sequence and/or a 3′ tailing sequence, e.g., a polyA tail. In other embodiments, the polynucleotides are expression cassettes comprising a promoter operatively linked to the coding sequences. In certain embodiments, the nucleic acid sequence encoding the multicomponent polypeptide molecule and the nucleic acid sequence encoding naturally occurring co-receptor ligand polypeptide are present in the same polynucleotide.

In further embodiments, pharmaceutical compositions comprising an expression vector, e.g., a viral vector, comprising a polynucleotide comprising a nucleic acid sequence encoding a multicomponent polypeptide molecule described herein and one or more pharmaceutically acceptable diluent, carrier, or excipient are also disclosed. In particular embodiments, the pharmaceutical composition further comprises an expression vector, e.g., a viral vector, comprising a polynucleotide comprising a nucleic acid sequence encoding a naturally occurring co-receptor ligand polypeptide. In certain embodiments, the nucleic acid sequence encoding the multicomponent polypeptide molecule and the nucleic acid sequence encoding the naturally occurring co-receptor ligand polypeptide are present in the same polynucleotide, e.g., expression cassette.

The present invention further contemplates a pharmaceutical composition comprising a cell comprising an expression vector comprising a polynucleotide comprising a promoter operatively linked to a nucleic acid encoding a multicomponent polypeptide molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a polypeptide corresponding to the natural ligand of the receptors. In particular embodiments, the cell is a heterologous cell or an autologous cell obtained from the subject to be treated. In particular embodiments, the cell is a stem cell, e.g., an adipose-derived stem cell or a hematopoietic stem cell.

The subject molecules, alone or in combination, can be combined with pharmaceutically-acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the multicomponent polypeptide molecule (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms are dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present invention includes pharmaceutically acceptable salts of a WNT multicomponent polypeptide molecule described herein. The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

In some embodiments, the pharmaceutical composition provided herein comprise a therapeutically effective amount of a WNT multicomponent polypeptide molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4 °C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

### IX. METHODS OF USE

For illustrative purposes only, the cell targeting multicomponent WNT molecule can be used as to treat various diseases or disorders where tissue regeneration is necessary. Such diseases include, but are not limited to: increase bone growth or regeneration, bone grafting, healing of bone fractures, treatment of osteoporosis and osteoporotic fractures, vertebral compression fractures, spinal fusion, osseointegration of orthopedic devices, tendon-bone integration, tooth growth and regeneration, dental implantation, periodontal diseases, maxillofacial reconstruction, and osteonecrosis of the jaw. Also contemplated are: treatment of alopecia; enhancing regeneration of sensory organs, e.g. treatment of hearing loss, including internal and external auditory hair cells, treatment of vestibular hypofunction, treatment of macular degeneration, treatment of vitreoretinopathy, diabetic retinopathy, other diseases of retinal degeneration, Fuchs' dystrophy, other cornea disease, etc.; treatment of stroke, traumatic brain injury, Alzheimer's disease, multiple sclerosis and other conditions affecting the blood brain barrier; treatment of spinal cord injuries, other spinal cord diseases. The compositions of this invention may also be used in treatment of oral mucositis, treatment of short bowel syndrome, inflammatory bowel diseases (IBD), other gastrointestinal disorders; treatment of metabolic syndrome, dyslipidemia, treatment of diabetes, treatment of pancreatitis, conditions where exocrine or endocrine pancreas tissues are damaged; conditions where enhanced epidermal regeneration is desired, e.g., epidermal wound healing, treatment of diabetic foot ulcers, syndromes involving tooth, nail, or dermal hypoplasia, etc., conditions where angiogenesis is beneficial; treatment of myocardial infarction, coronary artery disease, heart failure; enhanced growth of hematopoietic cells, e.g. enhancement of hematopoietic stem cell transplants from bone marrow, mobilized peripheral blood, treatment of immunodeficiencies, graft versus host diseases, etc.; treatment of acute kidney injuries, chronic kidney diseases; treatment of lung diseases, chronic obstructive pulmonary diseases (COPD), idiopathic pulmonary fibrosis (IPF) enhanced regeneration of lung tissues. The compositions of the present invention may also be used in enhanced regeneration of liver cells, e.g. liver regeneration, treatment of cirrhosis, enhancement of liver transplantations, treatment of acute liver failure, treatment of chronic liver diseases with hepatitis C or B virus infection or post-antiviral drug therapies, alcoholic liver diseases, alcoholic hepatitis, non-alcoholic liver diseases with steatosis or steatohepatitis, and the like. The compositions of this invention may treat diseases and disorders including, without limitation, conditions in which regenerative cell growth is desired.

In particular embodiments, a pharmaceutical composition is administered parenterally, e.g., intravenously, orally, rectally, or by injection. In some embodiments, it is administered locally, e.g., topically or intramuscularly. In some embodiments, a composition is administered to target tissues, e.g., to bone, joints, ear tissue, eye tissue, gastrointestinal tract, skin, a wound site or spinal cord. Methods of the invention may be practiced in vivo or ex vivo. In some embodiments, the contacting of a target cell or tissue with a multicomponent polypeptide molecule is performed ex vivo, with subsequent implantation of the cells or tissues, e.g., activated stem or progenitor cells, into the subject. The skilled artisan can determine an appropriate site of and route of administration based on the disease or disorder being treated.

The dose and dosage regimen may depend upon a variety of factors readily determined by a physician, such as the nature of the disease or disorder, the characteristics of the subject, and the subject's history. In particular embodiments, the amount of a multicomponent polypeptide molecule administered or provided to the subject is in the range of about 0.01 mg/kg to about 50 mg/kg, 0.1 mg/kg to about 500 mg/kg, or about 0.1 mg/kg to about 50 mg/kg of the subject's body weight.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular biology, cell biology and biochemistry can be found in such standard textbooks as “Molecular Cloning: A Laboratory Manual, 3rd Ed.” (Sambrook et al., Harbor Laboratory Press 2001); “Short Protocols in Molecular Biology, 4th Ed.” (Ausubel et al. eds., John Wiley & Sons 1999); “Protein Methods” (Bollag et al., John Wiley & Sons 1996); “Nonviral Vectors for Gene Therapy” (Wagner et al. eds., Academic Press 1999); “Viral Vectors” (Kaplift & Loewy eds., Academic Press 1995); “Immunology Methods Manual” (I. Lefkovits ed., Academic Press 1997); and “Cell and Tissue Culture: Laboratory Procedures in Biotechnology” (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

Recombinant molecules were generated that combine agonists for the WNT receptors, FZD and/or LRP co-receptors, together with bridging receptor, to create WNT signaling “cell targeting multicomponent polypeptide”

Materials and methods employed in the following Examples include the following.

Protein production: All recombinant proteins were produced in Expi293F cells (Thermo Fisher Scientific) by transient transfection unless otherwise specified. All IgG-based and Fc-containing constructs were first purified with Protein-A resin and eluted with 0.1 M glycine pH 3.5. All proteins were then polished by a size exclusion column in HBS buffer (10 mM HEPES pH 7.2, 150 mM NaCl). Proteins were supplemented with glycerol to 10% for long term storage at −80 °C.

SuperTop Flash (STF) assay: WNT signaling activity is measured using cell lines containing a luciferase gene controlled by a WNT-responsive promoter (Super Top Flash reporter assay, STF) as reported (Janda et al., 2017; Nature 545:234). In brief, cells are seeded at a density of 10,000 per well in 96-well plates 24 hr prior to treatment, then treated with RSPO or mimetic proteins overnight either alone or together with 30% WNT3a-conditioned media. WNT3a conditioned media are prepared from ATCC-CRL-2647 WNT3a secreting L cells following vendor recommended conditions. Cells were lysed with Luciferase Cell Culture Lysis Reagent (Promega) and activity are measured with Luciferase Assay System (Promega) using vendor suggested procedures. Data are plotted as average −/+standard deviation of triplicates and fitted by non-linear regression using Prism (GraphPad Software).

### Affinity Measurement and Step-Binding Assay

Binding kinetics of F-FGF21 series (F-FGF21FL, F-FGF21ΔN, F-FGF21ΔC, and F-FGF21ΔNΔC) to human FZD7 CRD and βKlotho (Fisher Scientific) or L-39F7 series (L-39F7, αGFP-39F7, and L-αGFP) to human LRP6E3E4 and βKlotho, respectively, were determined by bio-layer interferometry (BLI) using an Octet Red 96 (PALL ForteBio) instrument at 30 °C, 1000 rpm with AHC biosensors (Sartorius). Various F-FGF21 or L-39F7 proteins were diluted to 50 nM in the running buffer and captured to the AHC biosensor, followed by dipping into wells containing the FZD7 CRD, LRP6E3E4 and βKlotho at different concentrations in a running buffer or into a well with only the running buffer as reference channel. The dissociation of the interaction was followed with the running buffer. The monovalent KD for each binder was calculated by Octet System software, based on fitting to a 1:1 binding model.

Step-binding assay was performed with the BLI using the Octet Red 96 instrument at 30 °C, 1000 rpm with AHC biosensors. Various F-FGF21 or L-39F7 proteins were diluted to 50 nM in the running buffer and captured to the AHC biosensor, followed by dipping into wells containing the 100 nM FZD7 CRD or 100 nM LRP6E3E4, respectively. The sensor chips next moved into 150 nM βKlothos containing 100 nM FZD7 CRD or containing 100 nM LRP6E3E4 to check the additional bindings of β-Klotho. Sensorgram slopes were compared for βKlotho bindings.

### Primary Human Cells

Human hepatocytes were purchased from BioIVT (10-donor pooled cryoplateable X008001-P) and cultured in LONZA hepatocyte maintenance medium (CC-3198). In short, plastic culture plates were coated with 20% Matrigel Matrix (CB40230C) and cells were plated in plating medium (BioIVT Z990003). After four hours the medium was changed to maintenance medium and refreshed every day for three days prior to the 24 h experiment.

Human small intestinal organoids were a gift from the Calvin Kuo Lab at Stanford. Organoids were maintained and expanded as previously described32. In short, adapted expansion medium contained Advanced DMEM, 10 mM HEPES, 1× GlutaMAX, 1× Penicillin-Streptomycin, 1× B27, 1× N2, 1.25 mM N-acetylcysteine, 10 mM Nicotinamide, 50 ng/mL recombinant human EGF, 50 ng/mL recombinant human Noggin, 20 nM R-Spondin 2, 0.1 nML-F Wnt mimetic, 10 nM recombinant Gastrin, 500 nM A83-01 and 10 μM SB202190.

Treatment of molecules was done in the presence of 20 nM R-spondin 2 for 24 h at a concentration of 10 nM. After 24 hours the cells were harvested and RNA collected for qPCR. Each experiment with both primary human hepatocytes and human small intestinal organoids was repeated three times.

### Quantitative Polymerase Chain Reaction Analysis of Gene Expression

RNAs from HEK293, Huh7 cells, or primary human cells were extracted using the Qiagen RNeasy Micro Kit (Qiagen). cDNA was produced using the SuperScript IV VILO cDNA Synthesis Kit (Thermo Fisher). βKlotho (KLB) RNA was quantified using Maxima SYBR Green qPCR master mix on a Bio-Rad CFX96 real time PCR machine. Cycle threshold (Ct) values were normalized to the expression of constitutive ACTINB RNA using the following oligo's:

ACTB_F1:
(SEQ ID NO: 31)
CTGGAACGGTGAAGGTGACA;
ACTB_R1:
(SEQ ID NO: 32)
AAGGGACTTCCTGTAACAATGCA;
KLB_F1:
(SEQ ID NO: 33)
ATCTAGTGGCTTGGCATGGG;
KLB_R1:
(SEQ ID NO: 34)
CCAAACTTTCGAGTGAGCCTTG;
KLB_F2:
(SEQ ID NO: 35)
CACTGAATCTGTTCTTAAGCCCG;
KLB_R2:
(SEQ ID NO: 36)
GGCGTTCCACACGTACAGA;
KLB_F3:
(SEQ ID NO: 37)
GGAGGTGCTGAAAGCATACCT;
and
KLB_R3:
(SEQ ID NO: 38)
TCTCTTCAGCCAGTTTGAATGC.

### Example 1

### Multicomponent WNT Molecule for Concept of the 2:0/0:2 Split Design

To evaluate the structures shown in FIG. 1C, the βKlotho and endocrine fibroblast growth factor 21 (FGF21) ligand system were tested as the bridging receptors. FGF21 is an endocrine hormone produced by the liver that regulates metabolic homeostasis (BonDurant and Potthoff, 2018). FGF21 signals through FGFR1c, FGFR2c, and FGFR3c in the presence of co-receptor βKlotho (Zhang and Li, 2015). The binding of FGF21 to the receptor complex is primarily driven by its affinity toward βKlotho via its C-terminal domain (Lee et al., 2018, Shi et al., 2018), while its N-terminal domain is important for FGFR interaction and signaling (Yie et al., 2012, Micanovic et al., 2009). βKlotho binding antibodies have also been identified that could induce βKlotho/FGFR signaling, and one particular agonistic βKlotho antibody binds to a different epitope on βKlotho from FGF21 and does not compete with FGF21 binding (Min et al., 2018). Therefore, the following bridging receptor (βKlotho) binding elements were selected to test the cell targeting concept:
FGF21FL (full length FGF21) that can bind to βKlotho and is competent to induce FGFR signaling;
FGF21ΔC (FGF21 without the C-terminal βKlotho interaction domain) that does not bind βKlotho and is not capable of inducing FGFR signaling;
FGF21ΔN (FGF21 without the N-terminal FGFR interaction domain) that binds βKlotho but does not signal; FGF21ΔNΔC that cannot bind βKlotho and cannot signal; and
39F7 IgG that binds βKlotho and can induce FGFR signaling.

The FZD and LRP binding domains selected were F12578 (binds FZD1,2,5,7,8) and L (binds LRP6), previously named F1 and L2, respectively (Chen et al., 2020).

The graphic representations of the binders and the various combinations are shown in FIG. 2A-2C.

To test the structures described in FIG. 1C, the FZD binder (F12578) was combined with one of two versions of the bridging receptor (βKlotho) binder, F12578-FGF21FL or F12578-FGF21ΔN (FIG. 2B), and the LRP binder (L) was combined with the other bridging receptor (βKlotho) binder, 39F7, as L-39F7 (FIG. 2B). As shown in FIGS. 2D and 2E, the combination of F12578-FGF21FL or FGF12578-FGF21ΔN with L-39F7 resulted in WNT/β-catenin signaling in a liver cell line, Huh7 cells, which express the bridging receptor βKlotho, but not in 293 cells, where βKlotho is not expressed. This signaling depended on the presence of both the FZD and LRP binding arms and the ability to bind the bridging receptor, as the removal of LRP binding arm L from L-39F7 or inactivation of βKlotho binding (use of FGF21ΔNΔC) resulted in no activity in either cell type (FIGS. 2D and 2E). This provided experimental evidence supporting the concept presented in FIG. 1C.

Table 1 describes the different components/formats tested.

TABLE 1
Formats and sequences of multicomponent WNT polypeptide with 2:0/0:2 split design:
VH = Italic;
Human CH1CH2CH3 with LALAPG mutations (LALAPG mutations are only
marked in the first HC_IgG1_LALAPG) = Italic bold;
VL = Italic underlined:
Constant of lambda LC = Italic underlined dark gray; and
Constant of Kappa LC = Italic underlined bold;
Linker = light grey;
hFGF21ΔN = bold;
hFGF21FL= bold underline;
hFGF21ΔNΔC = light gray bold italic;
hFGF21ΔC = light gray italic; and
LALAPG mutations = bold dark gray underlined.

| | SEQ ID | |
|---|---|---|
| Structure | NO. | Sequences |
| L-39F7 LC | 1 | EVQLVESGGGLVQPGGSLRLSCAASGFTFTSYYISWVRQAPGKGLEWVAEISP |
| | | YSGSTYYADSVKGRFTISADTSK |
| | | 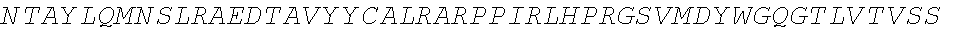 |
| | | 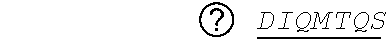 |
| | | PSSLSASVGDRVTITCRASQDVSTAVAWYQQKPGKAPKLLIYSASFLYSGVPSRF |
| | | SGSGSGTDFTLTISSLQPEDFA |
| | | 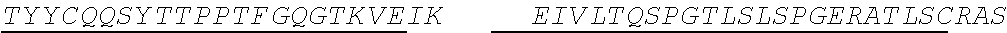 |
| | | QSVSSTYLAWYQQKPGQAPRLL |
| | | IYGASSRATGIPDRFSGSGSGTDFTLTISRLEPEDFAVYYCQQSGSSPLTFGGGT |
| | | EVEIKRTVAAPSVFIFPPSDEQL |
| | | KSGTASVVCLLNNFYPREAKVQWKVDNALQSGNSQESVTEQDSKDSTYS |
| | | LSSTLTLSKADYEKHKVYACEVTHQ |
| | | GLSSPVTKSFNRGEC |
| 39F7 HC | 2 | QVQLVESGGGVVQPGRSLRLSCAASGFTFSNYGIHWVRQAPGKGLEWVAVIW |
| | | YDGSIKYYADSVKGRFTISR |
| | | DNSKNTLYLQMNSLRAEDTAVYYCARDRAAAGLHYYYGMDVWGQGTTVTVSS |
| | | ASTKGPSVFPLAPSSKSTSG |
| | | GTAALGCLVKDYFPEPVTVSWNSGALTSGVHTFPAVLQSSGLYSLSSVVT |
| | | VPSSSLGTQTYICNVNHKPSNTKV |
| | | 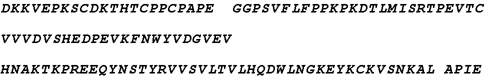 |
| | | KTISKAKGQPREPQVYTLPPSREEM |
| | | TKNQVSLTCLVKGFYPSDIAVEWESNGQPENNYKTTPPVLDSDGSFFLYS |
| | | KLTVDKSRWQQGNVFSCSVMHEA |
| | | LHNHYTQKSLSLSPGK |
| F12758- | 3 | DIELTQPPSVSVAPGQTARISCSGDNIGSFYVHWYQQKPGQAPVLVIYDKSNRP |
| FGF21FL | | |
| LC | | SGIPERFSGSNSGNTATLTISG |
| | | 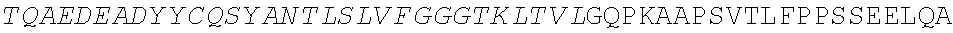 |
| | | 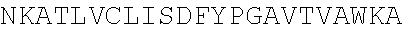 |
| | | 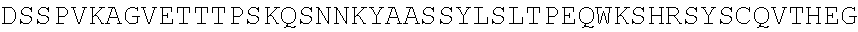 |
| | | 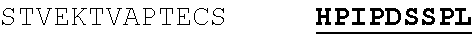 |
| | | LQFGGQVRQRYLYTDDAQQTEAHLEIREDGTVGGAADQSPESLLQL |
| | | KALKPGVIQILGVKTSRFLCQRPDGALYGSLHFDPEACSFRERLLED |
| | | GYNVYQSEAHGLPLHLPGNKSPHRDPAPRGPARFLPLPGLPPALPEP |
| | | PGILAPQPPDVGSSDPLSMVGGSQGRSPSYES |
| F12758 | 4 | EVQLVESGGGLVQPGGSLRLSCAASGFTFSHYTLSWVRQAPGKGLEWVSVISG |
| HC | | |
| | | DGSYTYYADSVKGRFTISSDN |
| | | SKNTLYLQMNSLRAEDTAVYYCARNFIKYVFANWGQGTLVTVSSASTKGPSVF |
| | | PLAPSSKSTSGGTAALGCLVKD |
| | | YFPEPVTVSWNSGALTSGVHTFPAVLQSSGLYSLSSVVTVPSSSLGTQTYI |
| | | CNVNHKPSNTKVDKKVEPKSCDKTHTCPPCPAPEAAGGPSVFLFPPKPK |
| | | DTLMISRTPEVTCVVVDVSHEDPEVKFNWYVDGVEVHNAKTKPREEQY |
| | | NSTYRVVSVLTVLHQDWLNGKEYKCKVSNKALGAPIEKTISKAKGQPREP |
| | | QVYTLPPSREEMTKNQVSLTCLVGFYPSDIAVEWESNGQPENNYKTTPPV |
| | | LDSDGSFFLYSKLTVDKSRWQQGNVFSCSVMHEALHNHYTQKSLSLSPG |
| | | K |
| F12758- | 5 | DIELTQPPSVSVAPGQTARISCSGDNIGSFYVHWYQQKPGQAPVLVIYDKSNRP |
| FGF21ΔN | | |
| LC | | SGIPERFSGSNSGNTATLTISGT |
| | | 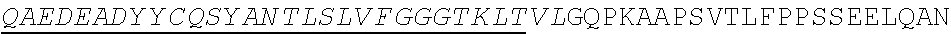 |
| | | 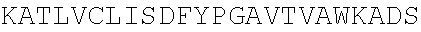 |
| | | 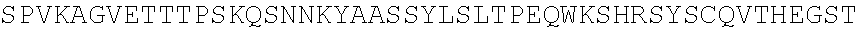 |
| | | 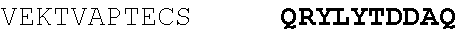 |
| | | QTEAHLEIREDGTVGGAADQSPESLLQLKALKPGVIQILGVKTSRFL |
| | | CQRPDGALYGSLHFDPEACSFRERLLEDGYNVYQSEAHGLPLHLPGN |
| | | KSPHRDPAPRGPARFLPLPGLPPALPEPPGILAPQPPDVGSSDPLSMV |
| | | GGSQGRSPSYES |
| L-αGFP | 6 | EVQLVESGGGLVQPGGSLRLSCAASGFTFTSYYISWVRQAPGKGLEWVAEISP |
| LC | | |
| | | YSGSTYYADSVKGRFTISADT |
| | | 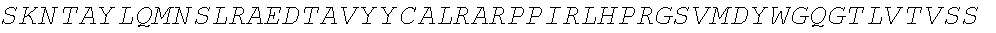 |
| | | 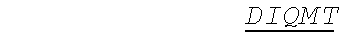 |
| | | QSPSSLSASVGDRVTITCRASQDVSTAVAWYQQKPGKAPKLLIYSASFLYSGVPS |
| | | RFSGSGSGTDFTLTISSLQPE |
| | | 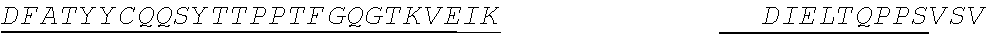 |
| | | APGQTARISCSGDNLGKKYVY |
| | | WYQQKPGQAPVLVIYGDDERPSGIPERFSGSNSGNTATLTISGTQAEDEADYY |
| | | CASYDSSHILIVFGGGTKLTVL |
| | | 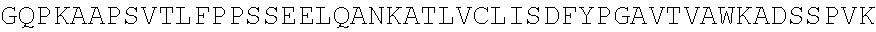 |
| | | 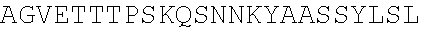 |
| | | 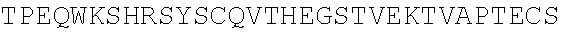 |
| αGFP HC | 7 | QVQLVESGGGLVQPGGSLRLSCAASGFTFSRYGMHWVRQAPGKGLEWVSGIS |
| | | SIGSNTYYADSVKGRFTISRD |
| | | NSKNTLYLQMNSLRAEDTAVYYCARWYKTYIDVWGQGTLVTVSSASTKGPSV |
| | | FPLAPSSKSTSGGTAALGCLVK |
| | | DYFPEPVTVSWNSGALTSGVHTFPAVLQSSGLYSLSSVVTVPSSSLGTQTY |
| | | ICNVNHKPSNTKVDKKVEPKSCDK |
| | | THTCPPCPAPEAAGGPSVFLFPPKPKDTLMISRTPEVTCVVVDVSHEDPE |
| | | VKFNWYVDGVEVHNAKTKPREEQ |
| | | YNSTYRVVSVLTVLHQDWLNGKEYKCKVSNKALGAPIEKTISKAKGQPRE |
| | | PQVYTLPPSREEMTKNQVSLTCLVK |
| | | GFYPSDIAVEWESNGQPENNYKTTPPVLDSDGSFFLYSKLTVDKSRWQQ |
| | | GNVFSCSVMHEALHNHYTQKSLSLSPGK |
| F12758- | 8 | DIELTQPPSVSVAPGQTARISCSGDNIGSFYVHWYQQKPGQAPVLVIYDKSNRP |
| FGF21AC | | |
| LC | | SGIPERFSGSNSGNTATLTISGT |
| | | 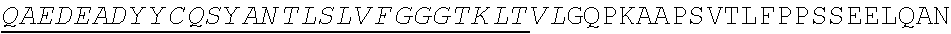 |
| | | 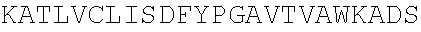 |
| | | 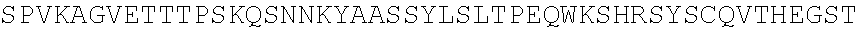 |
| | | 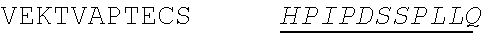 |
| | | FGGQVRQRYLYTDDAQQTEAHLEIREDGTVGGAADQSPESLLQLKALKP |
| | | GVIQILGVKTSRFLCQRPDGALYGSLH |
| | | FDPEACSFRERLLEDGYNVYQSEAHGLPLHLPGNKSPHRDPAPRGPARFL |
| | | PLPGLPPALPEPPGILAPQPPDVG |
| F12758- | 9 | DIELTQPPSVSVAPGQTARISCSGDNIGSFYVHWYQQKPGQAPVLVIYDKSNRP |
| FGF21ANA | | |
| CLC | | SGIPERFSGSNSGNTATLTISG |
| | | 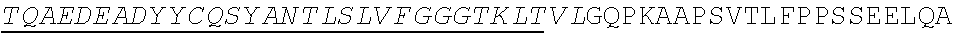 |
| | | 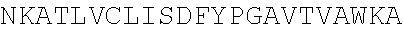 |
| | | 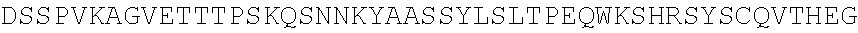 |
| | | 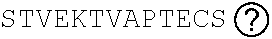 |
| | | 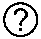 |
| | | 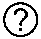 |
| | | 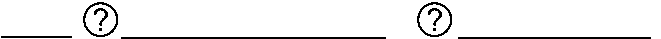 |
| | | 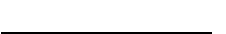 |

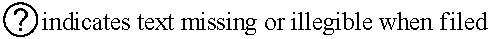

### Example 2

### Multicomponent WNT Molecule for Concept of the 1:1 Split Design

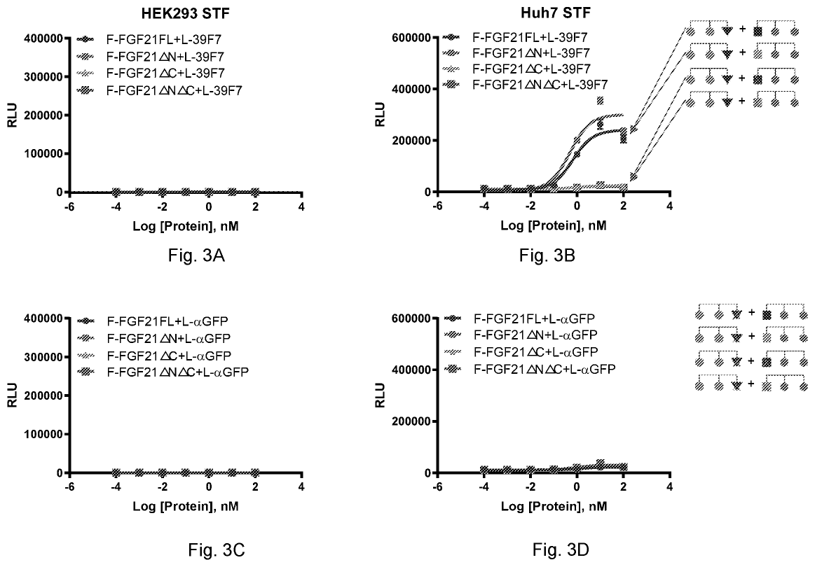

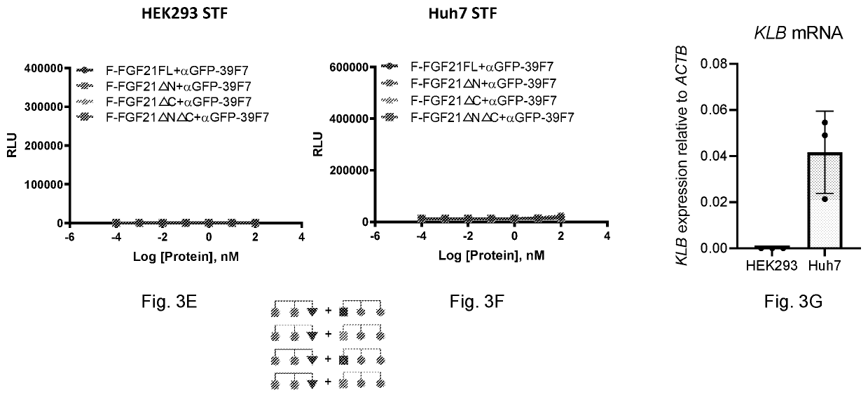

To test the concept depicted in FIG. 1G, the FZD binder (F12578), LRP binder (L), and the two non-competing bridging receptor (βKlotho) binders (FGF21FL and 39F7) were combined in the molecule, F12578-FGF21FL-39F7-L, depicted in FIG. 3A. As shown in FIG. 3B, F12578-FGF21FL-39F7-L activated WNT/β-catenin signaling in Huh7 cells that express the bridging receptor βKlotho but not in 293 cells where βKlotho is not expressed (FIG. 3B). No activity was observed in 293 cells. This provided validation for the concept presented in FIG. 1G. Table 2 describes the different components/formats tested.

TABLE 2
Formats and sequences of multicomponent WNT polypeptide with 1:0/01: split design:
VH = Italic;
Human CH1CH2CH3 with LALAPG mutations (LALAPG mutations are only marked in the
first HC_IgG1_LALAPG) = Italic bold;
VL = Italic underlined;
Constant of lambda LC = dark gray;
Constant of Kappa LC = Italic underlined bold;
G4S linker = light grey;
hFGF21ΔN =bold;
hFGF21FL=bold underline;
hFGF21ΔNΔC = light gray bold italic;
hFGF21ΔC = light gray italic;
Amino acids mutations in SEQ 11 and SEQ 12 = bold dark gray underlined.

| Structure | SEQ ID NO. | Sequences |
|---|---|---|
| 39F7 LC | 10 | EIVLTQSPGTLSLSPGERATLSCRASQSVSSTYLAWYQQKPGQAPRLLIYGASSRATGIP |
| | | DRFSGSGSGTDFTLTISRLEPEDFAVYYCQQSGSSPLTFGGGTEVEIKRTVAAPSVF |
| | | IFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC |
| F12758_ | 11 | EVQLVESGGGLVQPGGSLRLSCAASGFTFSHYTLSWVRQAPGKGLEWVSVISGDGSYT |
| Hole_ | | |
| Flag HC | | YYADSVKGRFTISSDNSKNTLYLQMNSLRAEDTAVYYCARNFIKYVFANWGQGTLVTVS |
| | | SASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSCDKTHTCP |
| | | PCPAPEAAGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKALGAPIEKT |
| | | 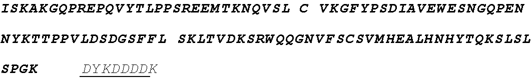 |
| | | T366S L368A Y407V are mutations for the “Hole” of Knob and Hole |
| 39F7_ | 12 | QVQLVESGGGVVQPGRSLRLSCAASGFTFSNYGIHWVRQAPGKGLEWVAVIWYDGSI |
| Knob _HC | | |
| | | KYYADSVKGRFTISRDNSKNTLYLQMNSLRAEDTAVYYCARDRAAAGLHYYYGMDVW |
| | | GQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKKVEPKS |
| | | CDKTHTCPPCPAPEAAGGPSVFLFPPKPKDTLMISRTPEVTCVVVDVSHEDPEV |
| | | KFNWYVDGV |
| | | EVHNAKTKPREEQYNSTYRVVSVLTVLHQDWLNGKEYKCKVSNKALGAPIEKTI |
| | | 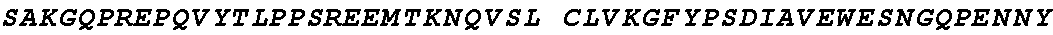 |
| | | KTTPPVLDSDGSFFLYSKLTVDKSRWQQGNVFSCSVM |
| | | HEALHNHYTQKSLSLSPGK |
| | | T366W is the “Knob” of Knob and Hole |
| a GFP- | 13 | QVQLVESGGGLVQPGGSLRLSCAASGFTFSRYGMHWVRQAPGKGLEWVSGISSIGSN |
| 39F7 LC | | |
| | | TYYADSVKGRFTISRDNSKNTLYLQMNSLRAEDTAVYYCARWYKTYIDVWGQGTLVTV |
| | | 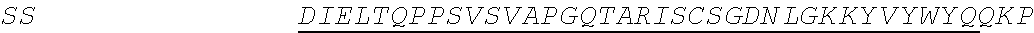 |
| | | GQAPVLVIYGDDERPSGIPERFSGSNSGNTATLTISGTQAEDEADYYCASYDSSHILIVF |
| | | 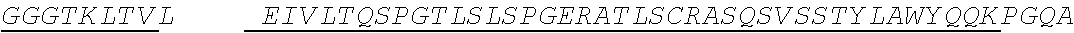 |
| | | PRLLIYGASSRATGIPDRFSGSGSGTDFTLTISRLEPEDFAVYYCQQSGSSPLTFGGGTE |
| | | VEIKRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSENRGE |
| | | C |
| F12758- | 3 | DIELTQPPSVSVAPGQTARISCSGDNIGSFYVHWYQQKPGQAPVLVIYDKSNRPSGIPE |
| FGF21FL | | |
| LC | | 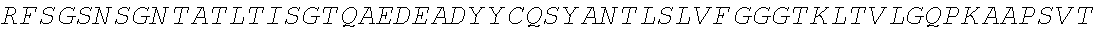 |
| | | 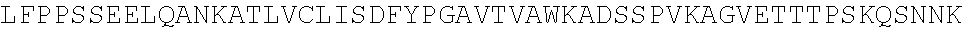 |
| | | 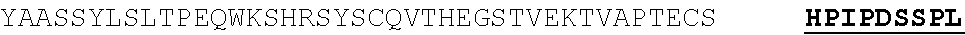 |
| | | LQFGGQVRQRYLYTDDAQQTEAHLEIREDGTVGGAADQSPESLLQLKALK |
| | | PGVIQILGVKTSRFLCQRPDGALYGSLHFDPEACSFRERLLEDGYNVYQSEA |
| | | HGLPLHLPGNKSPHRDPAPRGPARFLPLPGLPPALPEPPGILAPQPPDVGSSD |
| | | PLSMVGGSQGRSPSYES |

The various embodiments described above can be combined to provide further embodiments. All of the U.S. patents, U.S. patent application publications, U.S. patent applications, foreign patents, foreign patent applications, and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments. These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### REFERENCES

1. Dickopf, S., Georges, G. J. & Brinkmann, U. Format and geometries matter: Structure-based design defines the functionality of bispecific antibodies. Comput Struct Biotechnol J 18, 1221-1227 (2020).
2. Cironi, P., Swinburne, I. A. & Silver, P. A. Enhancement of cell type specificity by quantitative modulation of a chimeric ligand. J Biol Chem 283, 8469-8476 (2008).
3. Taylor, N. D., Way, J. C., Silver, P. A. & Cironi, P. Anti-glycophorin single-chain Fv fusion to low-affinity mutant erythropoietin improves red blood cell-lineage specificity. Protein Eng Des Sel 23, 251-260 (2010).
4. Burrill, D. R., Vernet, A., Collins, J. J., Silver, P. A. & Way, J. C. Targeted erythropoietin selectively stimulates red blood cell expansion in vivo. Proc Natl Acad Sci USA 113, 5245-5250 (2016).
5. Garcin, G. et al. High efficiency cell-specific targeting of cytokine activity. Nat Commun 5, 3016 (2014).
6. Ghasemi, R. et al. Selective targeting of IL-2 to NKG2D bearing cells for improved immunotherapy. Nat Commun 7, 12878 (2016).
7. Lazear, E. et al. Targeting of IL-2 to cytotoxic lymphocytes as an improved method of cytokine-driven immunotherapy. Oncoimmunology 6, e1265721 (2017).
8. Puskas, J. et al. Development of an attenuated interleukin-2 fusion protein that can be activated by tumour-expressed proteases. Immunology 133, 206-220 (2011).
9. Skrombolas, D., Sullivan, M. & Frelinger, J. G. Development of an Interleukin-12 Fusion Protein That Is Activated by Cleavage with Matrix Metalloproteinase 9. J Interferon Cytokine Res 39, 233-245 (2019).
10. Banaszek, A. et al. On-target restoration of a split T cell-engaging antibody for precision immunotherapy. Nat Commun 10, 5387 (2019).
11. Mock, J. et al. An engineered 4-1BBL fusion protein with “activity on demand”. Proc Natl Acad Sci US A 117, 31780-31788 (2020).
12. Quijano-Rubio, A. et al. A split, conditionally active mimetic of IL-2 reduces the toxicity of systemic cytokine therapy. Nat Biotechnol (2022).
13. Nusse, R. & Clevers, H. Wnt/beta-Catenin Signaling, Disease, and Emerging Therapeutic Modalities. Cell 169, 985-999 (2017).
14. Janda, C. Y., Waghray, D., Levin, A. M., Thomas, C. & Garcia, K. C. Structural basis of Wnt recognition by Frizzled. Science 337, 59-64 (2012).
15. Kadowaki, T., Wilder, E., Klingensmith, J., Zachary, K. & Perrimon, N. The segment polarity gene porcupine encodes a putative multitransmembrane protein involved in Wingless processing. Genes Dev 10, 3116-3128 (1996).
16. Dijksterhuis, J. P. et al. Systematic mapping of WNT-FZD protein interactions reveals functional selectivity by distinct WNT-FZD pairs. J Biol Chem 290, 6789-6798 (2015).
17. Janda, C. Y. et al. Surrogate Wnt agonists that phenocopy canonical Wnt and beta-catenin signalling. Nature 545, 234-237 (2017).
18. Chen, H. et al. Development of Potent, Selective Surrogate WNT Molecules and Their Application in Defining Frizzled Requirements. Cell Chem Biol 27, 598-609 e594 (2020).
19. Tao, Y. et al. Tailored tetravalent antibodies potently and specifically activate Wnt/Frizzled pathways in cells, organoids and mice. Elife 8 (2019).
20. Miao, Y. et al. Next-Generation Surrogate Wnts Support Organoid Growth and Deconvolute Frizzled Pleiotropy In Vivo. Cell Stem Cell 27, 840-851 e846 (2020).
21. Xie, L. et al. Robust colonic epithelial regeneration and amelioration of colitis via FZD-specific activation of Wnt signaling. Cell Mol Gastroenterol Hepatol (2022).
22. BonDurant, L. D. & Potthoff, M. J. Fibroblast Growth Factor 21: A Versatile Regulator of Metabolic Homeostasis. Annu Rev Nutr 38, 173-196 (2018).
23. Zhang, J. & Li, Y. Fibroblast Growth Factor 21 Analogs for Treating Metabolic Disorders. Front Endocrinol (Lausanne) 6, 168 (2015).
24. Lee, S. et al. Structures of beta-klotho reveal a ‘zip code’-like mechanism for endocrine FGF signalling. Nature 553, 501-505 (2018).
25. Shi, S. Y. et al. A systematic dissection of sequence elements determining beta-Klotho and FGF interaction and signaling. Sci Rep 8, 11045 (2018).
26. Yie, J. et al. Understanding the physical interactions in the FGF21/FGFR/beta-Klotho complex: structural requirements and implications in FGF21 signaling. Chem Biol Drug Des 79, 398-410 (2012).
27. Micanovic, R. et al. Different roles of N- and C-termini in the functional activity of FGF21. J Cell Physiol 219, 227-234 (2009).
28. Min, X. et al. Agonistic beta-Klotho antibody mimics fibroblast growth factor 21 (FGF21) functions. J Biol Chem 293, 14678-14688 (2018).
29. Zhang, Z. et al. Tissue-targeted R-spondin mimetics for liver regeneration. Sci Rep 10, 13951 (2020).
30. Lo, M. et al. Effector-attenuating Substitutions That Maintain Antibody Stability and Reduce Toxicity in Mice. J Biol Chem 292, 3900-3908 (2017).
31. Stanislaus, S. et al. A Novel Fc-FGF21 With Improved Resistance to Proteolysis, Increased Affinity Toward beta-Klotho, and Enhanced Efficacy in Mice and Cynomolgus Monkeys. Endocrinology 158, 1314-1327 (2017).
32. Sato, T. et al. Long-term expansion of epithelial organoids from human colon, adenoma, adenocarcinoma, and Barrett's epithelium. Gastroenterology 141, 1762-1772 (2011).

What is claimed is:

1. A cell targeting multicomponent polypeptide molecule comprising:
a) at least one first antigen binding domain that binds to a first signaling receptor component,
b) at least one second antigen binding domain that binds to a second signaling receptor component, and
c) at least one different antigen binding domain that binds to a bridging molecule,
wherein the first signaling receptor and second signaling receptor can be different or identical.

2. The cell targeting multicomponent polypeptide molecule of claim 1, wherein the binding domain that binds to the bridging molecule comprises at least two binding domains that:
a) bind to different epitopes on the bridging molecule; or
b) bind to the same epitope on the bridging molecule.

3. The cell targeting multicomponent polypeptide molecule of claim 1 or claim 2, wherein
a) the first antigen binding domain that binds to a first signaling receptor component is tethered to at least one binding domain that binds to the bridging molecule; and
b) the second antigen binding domain that binds to a second signaling receptor component is tethered to at least one binding domain that binds to the bridging molecule.

4. The multicomponent polypeptide molecule of claim 3, wherein the antigen binding domain is joined directly or by a linker to the binding domain that binds to the bridging molecule.

5. The multicomponent polypeptide molecule of claim 3, wherein the bridging molecule is monomeric or multimeric.

6. The multicomponent polypeptide molecule of claim 3, wherein the binding domains are each independently selected from the group consisting of: an scFv, a V_{HH}/sdAb, a Fab, and a Fab′2.

7. The multicomponent polypeptide molecule of claim 3, wherein the binding domains are joined through a linker by a peptide linker comprising from 1-100 amino acids.

8. The multicomponent polypeptide molecule of claim 3, wherein the binding domains are attached to the N-terminus of an antibody Fc domain.

9. A nucleic acid encoding the multicomponent polypeptide molecule of any one of claims 1-8 or a polypeptide component thereof.

10. A vector comprising the nucleic acid of claim 9, wherein the vector is an expression vector comprising a promoter sequence operatively linked to the nucleic acid sequence; optionally wherein the vector is a virus comprising a promoter operatively linked to the nucleic acid.

11. A host cell comprising the vector of claim 10.

12. A process for producing the multicomponent polypeptide molecule of any one of claims 1-8 or a polypeptide component thereof, comprising culturing the host cell of claim 11 under conditions wherein the multicomponent polypeptide molecule or polypeptide component thereof is expressed by the expression vector.

13. A multicomponent WNT molecule comprising:
a) at least one FZD binding domain;
b) at least one LRP binding domain; and
c) at least one bridging molecule,
wherein the multicomponent WNT multicomponent polypeptide molecule modulates WNT signaling.

14. The multicomponent WNT molecule of claim 13, comprising:
a) one or two FZD binding domains tethered to the bridging molecule by a binding domain specific for a first epitope on the bridging molecule; and
b) one or two LRP binding domains tethered to the bridging molecule by a binding domain specific for a second epitope on the bridging molecule,
wherein the multicomponent WNT molecule is capable of activating WNT signaling.

15. The multicomponent WNT molecule of claim 13 or claim 14, wherein:
a) the binding domain specific for the first epitope of the bridging molecule and the FZD binding domain(s) are joined directly or joined by a linker;
the binding domain specific for the second epitope of the bridging molecule and the LRP binding domain(s) are joined directly, or joined by a linker; and
c) the binding domain specific for the first epitope and the binding domain specific for the second epitope of the bridging molecule are identical or different.

16. The multicomponent WNT molecule of claim 15, wherein the linker is a peptide or non-peptide linker.

17. The multicomponent WNT molecule of claim 13, wherein the FZD binding domain(s) and the LRP binding domain(s) are each independently selected from the group consisting of: a scFv, a V_{HH}/sdAb, a Fab, and a Fab′2.

18. The multicomponent WNT molecule of claim 13, wherein the FZD binding domain(s) and the LRP binding domains(s) are joined by a peptide linker comprising from 1-100 amino acids.

19. The multicomponent WNT molecule of claim 13, wherein the FZD binding domain(s) and the LRP binding domain(s) are attached to the N-terminus of an antibody Fc domain.

20. A nucleic acid encoding the multicomponent WNT molecule of any one of claims 13-19 or a polypeptide component thereof.

21. A vector comprising the nucleic acid of claim 20, wherein the vector is an expression vector comprising a promoter sequence operatively linked to the nucleic acid sequence; optionally wherein the vector is a virus comprising a promoter operatively linked to the nucleic acid.

22. A host cell comprising the vector of claim 21.

23. A process for producing the multicomponent WNT molecule or polypeptide component thereof of claim 13, comprising culturing the host cell of claim 22 under conditions wherein the multicomponent WNT protein or polypeptide component thereof is expressed by the expression vector.

24. A pharmaceutical composition to modulate the WNT/β-catenin signaling pathway comprising:
a) an effective amount of the multicomponent WNT molecule of any one of claims 13-19; and
b) a pharmaceutically acceptable diluent, adjuvant, or carrier.

25. A method of treating or preventing a disease or disorder in a subject in need thereof, the method comprising providing to the subject an effective amount of the multicomponent WNT molecule of any one of claims 13-19 or the pharmaceutical composition of claim 24.

26. A multicomponent WNT molecule comprising:
a) at least one FZD binding domain linked to one LRP binding domain which is tethered to the bridging molecule by a binding domain specific for a first epitope on the bridging molecule; and
b) at least one FZD binding domain linked to one LRP binding domain which is tethered to the bridging molecule by a binding domain specific for a second epitope on the bridging molecule;
wherein the tethering of the FZD and LRP binding domains to the bridging receptor forms a WNT mimetic capable of activating WNT signaling.

27. The multicomponent WNT molecule of claim 26, wherein:
a) the binding domain specific for a first epitope of the bridging molecule and the FZD/LRP binding domains are joined directly or by a linker;
b) the binding domain specific for a second epitope of the bridging molecule and the FZD/LRP binding domains are joined directly, or by a linker;
c) the binding domain of the first epitope and second epitope of the bridging molecule are identical or different; and/or
d) the binding domain of the first epitope and the second epitope of the bridging molecule could be linked together to FZD/LRP.

28. The multicomponent WNT molecule of claim 26 or claim 27, wherein the bridging molecule is monomeric or multimeric.

29. The multicomponent WNT molecule of any one of claims 26-28, wherein at least one of the FZD or LRP binding domains is selected from the group consisting of: a scFv, a VHH/sdAb, a Fab, and a Fab′2.

30. The multicomponent WNT molecule of any one of claims 26-29, wherein the binding domains are joined through a linker by a peptide link comprising from 1-100 amino acids.

31. The multicomponent WNT molecule of any one of claims 26-30, wherein the binding domains are attached to the N-terminus of an antibody Fc domain.

32. A nucleic acid encoding the multicomponent WNT molecule of any one of claims 26-31 or a polypeptide component thereof.

33. A vector comprising the nucleic acid of claim 32, wherein the vector is an expression vector comprising a promoter sequence operatively linked to the nucleic acid sequence; optionally wherein the vector is a virus comprising a promoter operatively linked to the nucleic acid.

34. A host cell comprising the vector of claim 33.

35. A process for producing the multicomponent WNT molecule or component thereof of any one of claims 26-31, comprising culturing the host cell of claim 36 under conditions wherein multicomponent WNT or polypeptide component thereof is expressed by the expression vector.

36. A pharmaceutical composition to modulate the WNT/β-catenin signaling pathway comprising:
a) an effective amount of the multicomponent WNT of any one of claims 26-31; and
b) a pharmaceutically acceptable diluent, adjuvant, or carrier.

37. A method of treating or preventing a disease or disorder in a subject in need thereof, the method comprising providing to the subject an effective amount of the multicomponent WNT molecule of any one of claims 26-31 or the pharmaceutical composition of claim 36.
